(19) United States
(12) Patent Application Publication
(10) Pub. No.: US 2012/0258047 A1
(43) Pub. Date: Oct. 11, 2012

(54) EPSILON-POLY-LYSINE CAPSULES

(76) Inventors: Christopher John Martoni, Montreal (CA); Mitchell Lawrence Jones, Montreal (CA); Satya Prakash, Montreal (CA)

(21) Appl. No.: 13/514,745

(22) PCT Filed: Dec. 22, 2010
(86) PCT No.: PCT/CA10/02057

§ 371 Date: Jun. 8, 2012

Related U.S. Application Data
(60) Provisional application No. 61/289,727, filed on Dec. 23, 2009.

(51) Int. Cl. A61K 9/14; A61K 33/44; A61K 49/00; C12P 1/00; A61K 38/43; A61K 38/02; C12N 1/00; A61K 35/74; A61K 35/66; B82Y 5/00

(52) U.S. Cl. 424/9.1; 424/491; 424/93.4; 424/125; 424/93.1; 435/41; 424/94.1; 514/1.1; 435/243; 977/742; 977/700

(57) ABSTRACT

The present disclosure relates to capsules having a core and one or more capsular walls surrounding the core, wherein at least one of the capsular walls comprises a polymer comprising epsilon-poly-lysine or a derivative thereof.

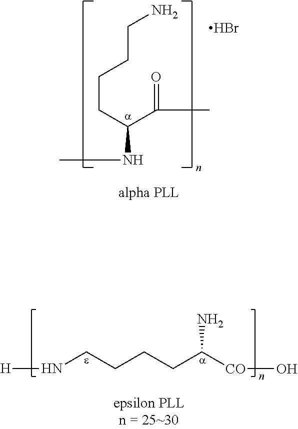

### RELATED APPLICATIONS

This application claims the benefit of priority to U.S. provisional application No. 61/289,727 filed Dec. 23, 2009, and PCT/CA2010/000660 filed Apr. 30, 2010, the contents of which are incorporated herein by reference in their entirety.

### FIELD OF THE DISCLOSURE

The present disclosure relates to a capsule comprising a core and a capsular wall comprising epsilon(ε)-poly-lysine or a derivative thereof.

### BACKGROUND OF THE DISCLOSURE

Encapsulation and immobilization patents include U.S. Pat. No. 6,565,777, U.S. Pat. No. 6,346,262, U.S. Pat. No. 6,258,870, U.S. Pat. No. 6,264,941, U.S. Pat. No. 6,217,859, U.S. Pat. No. 5,766,907 and U.S. Pat. No. 5,175,093. Artificial cell microencapsulation is a technique used to encapsulate biologically active materials in specialized ultra thin semi-permeable polymer membranes.^{1,2} The polymer membrane protects encapsulated materials from harsh external environments, while at the same time allowing for the metabolism of selected solutes capable of passing into and out of the microcapsule. In this manner, the enclosed material is retained inside and separated from the external environment, making microencapsulation particularly useful for biomedical and clinical applications.^{3,4,8} Studies show that artificial cell microcapsules can be used for oral administration of live genetically engineered cells that can be useful for therapeutic functions.^{6,7} Examples of applications of microencapsulation of enzymes, cells and genetically engineered microorganisms are xanthine oxidase for Lesch-Nyhan disease; phenylalanine ammonia lyase for phenylketonuria and E. coli DH5 cells for lowering urea, ammonia and other metabolites.⁸ Although the live cells remain immobilized inside the microcapsules, microencapsulation does not appear to hinder their growth kinetics.⁹ The microcapsules remain intact during passage through the intestinal tract and are excreted intact with the stool in about 24 hours. The cells are retained inside, and excreted with, the intact microcapsules addressing many of the major safety concerns associated with the use of live bacterial cells for various clinical applications. The membranes of the microcapsules are permeable to smaller molecules, and thus the cells inside the microcapsules metabolize small molecules found within the gut during passage through the intestine.^{1,6,7,9,10,11}

In 1980, Lim and Sum introduced the alginate-poly-l-lysine-alginate (APA) membrane system for islet encapsulation using α-poly-l-lysine.¹² Alginate beads are first produced by the extrusion of alginate droplets into millimolar concentrations of calcium or barium ions, thus forming a gel matrix around the biological material. The mild yet sturdy properties of the gel-like matrix allow the cell to remain viable with retention of enzymatic activity for extended periods of time. Binding of α-poly-L-lysine to alginate occurs electrostatically by long-chain alkyl amino groups that extend from the polyamide backbone of α-poly-L-lysine and interact with carboxyl groups of the calcium alginate bead.

Membrane thickness is known to correlate with permeability, resistance, mechanical strength, drug release capacity, and biocompatibility and alginate-α-poly-lysine-alginate capsules have been shown to have increased membrane thickness and mechanical strength. APA capsules have been shown to significantly prevent bacteria release into the medium and limit bacteria on the surface of capsules as compared to immobilized cultures.

APA microcapsules have been used to encase mammalian cells, bacterial cells, enzymes, etc. with several advantages noted over traditional immobilization technologies. α-Poly-l-lysine provides a perm-selective layer that can be quantified for mass transport and controlled by adjusting reaction time and concentration.¹³ Since charged α-poly-L-lysine is known to be immunogenic, re-exposure of the cross-linked bead to dilute alginate is used to neutralize the capsule surface, thus forming the APA membrane.¹⁴ However, studies suggest that the external alginate coating does not effectively neutralize the immunogenic α-poly-l-lysine. As a result, several studies have reported that APA microcapsules are not optimally biocompatible since they activate complement as well as IL-1 and TNF-a production by macrophages.¹⁵ In one study, intra-peritoneal implanted alginate beads elicited a less severe pericapsular reaction than complete APA microcapsules.¹⁶ In addition, the high cost of α-poly-L-lysine is a drawback for producing such capsules.

Organisms as evolutionally distant as bacteria and fungi have been reported to secrete ε-polylysine during fermentation²⁶⁻²⁸. Many bacteria, yeasts, moulds, and plants likely produce the polyamino acid. Culture media obtained through fermentation of a variety of microorganisms can be used in one of several biotechnological processes, wherein a stepwise treatment and purification process is initiated to provide the desired ε-polylysine homopolymer²⁶⁻²⁵. Differences in the genera, species, and strain utilized for fermentation, as well as the biotechnological processing itself may result in a homopolymer of c-polylysine with a wide range of molecular weights, chain lengths and biochemical properties. For instance, ε-polylysine produced by strains of the species S. albulus generally consist of 25-35 lysine residues while ε-polylysine of shorter lengths have been reported in several other strains^{26,28}. Also, the production of chemically modified ε-polylysine derivatives has been reported²⁶ and post-translational modification of the polyamino acid may lead to other derivatives and combinations of ε-polylysine species.

### SUMMARY OF THE DISCLOSURE

The present disclosure relates to a capsule comprising a core and a capsular wall comprising ε-poly-lysine or a derivative thereof. In particular, the capsule comprises;
i) a core, comprising an active ingredient encapsulated by a first polymer; and
ii) one or more capsular walls surrounding the core, wherein at least one of the capsular walls comprises a second polymer comprising ε-poly-lysine or a derivative thereof.

In another embodiment, the ε-poly-lysine or derivative thereof is produced from the fermentation of yeast or bacteria. In a further embodiment, the ε-poly-lysine produced from the fermentation yeast or bacteria is post-translationally modified.

In another embodiment, the polymer comprising ε-poly-lysine or a derivative thereof comprises a polymer of the formula (I)

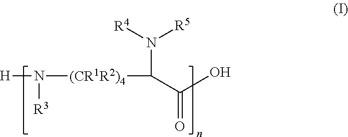

wherein

R¹ and R² are independently or simultaneously selected from H, halo, a lipid, a carbohydrate, a phosphate, an acetate group, (C₁-C₁₀)-alkyl, (C₁-C₁₀)-alkoxy, (C₂-C₁₀)-alkenyl, (C₂-C₁₀)-alkynyl, (C₃-C₁₀)-cycloalkyl phenyl, wherein the latter six groups are optionally substituted,

R³ is selected from H, (C₁-C₆)-alkyl, (C₂-C₆)-alkenyl and (C₂-C₆)-alkynyl,

R⁴ and R⁵ are independently selected from H, (C₁-C₁₀)-alkyl, (C₂-C₁₀)-alkenyl, (C₂-C₁₀)-alkynyl, (C₃-C₁₀)-cycloalkyl and phenyl, wherein the latter six groups are optionally substituted,

n is an integer from 1 to 50,

the optional substituents are selected from one to five of halo, (C₁₋₆)-alkyl and fluorosubstituted (C₁₋₆)-alkyl,

and all stereoisomers and enantiomers thereof.

In an embodiment, the polymer comprising ε-poly-lysine or derivative thereof is a polymer of the formula (ε-poly-L-lysine):

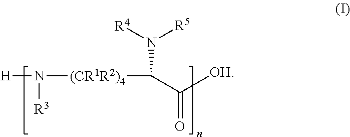

The present disclosure also includes a method for delivering an active ingredient to the gastrointestinal system of an animal comprising orally delivering a capsule according to the present disclosure to the animal. In another embodiment, there is also included a method for delivering probiotic organisms to the gastrointestinal system of an animal comprising orally delivering a capsule according to the present disclosure to the animal. In another embodiment, there is also included a method for transplanting eukaryotic cells into an animal comprising transplanting a capsule according to the present disclosure into the animal. In another embodiment, there is also included a method for delivering vectors to the gastrointestinal system of an animal comprising orally delivering a capsule according to the present disclosure into the gastrointestinal system of the animal. In another embodiment, there is also included a method for haemoperfusion of an animal wherein the haemoperfusion device contains a capsule according to the present disclosure. In another embodiment, there is also included a method for the fermentation of a substrate, comprising contacting the substrate with a capsule of the disclosure, wherein the capsule comprises microorganisms which ferment the substrate. In another embodiment, there is included a method for delivery of a carbon nanotube to an animal comprising orally delivering a capsule according to the present disclosure containing the carbon nanotube to the animal. In another embodiment, the carbon nanotube contains a pharmaceutical agent, which is released from the nanotube upon administration to the animal.

In another embodiment of the disclosure, there is also included a method for preventing, or reducing, phage attack of a fermentation organism during a fermentation process, wherein the fermentation process comprises:

(i) contacting a substrate with a capsule as defined herein, wherein the active ingredient comprises a fermentation organism, and

(ii) fermenting the substrate,

wherein the capsule protects the fermentation organism from phage attack.

In another embodiment, the capsules of the disclosure have an improved buoyancy, or a decreased tendency, to settle in a liquid.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the following drawings in which:

FIG. 1 shows the chemical structure of alpha (α) and epsilon (ε) polylysine;

FIG. 2 are light photomicrographs of alginate-α-polylysine-alginate (AαPA) (left) and alginate-ε-polylysine-alginate (AεPA) (right) microcapsules using an oil emersion lens at 1000× magnification in an embodiment of the disclosure;

FIG. 3 are transition electron photomicrographs of alginate-α-polylysine-alginate (AαPA) and alginate-ε-polylysine-alginate (AεPA) microcapsule membranes, in an embodiment of the disclosure, where the top row shows the AαPA microcapsule membrane at ascending magnification from left to right (6,000×, 43,000×, and 220,000×) and the bottom row shows the AεPA microcapsule membrane at ascending magnification from left to right (6,000×, 43,000×, and 220,000×). The reference line in each micrograph measures 2 μm, 0.2 μm, and 100 nm from left to right, respectively;

FIG. 4 are scanning electron photomicrographs of alginate-α-polylysine-alginate (AαPA) and alginate-ε-polylysine-alginate (AεPA) microcapsule membranes, respectively.

FIG. 5 is a graph illustrating the viability of Lactobacillus reuteri NCIMB 701089 cells grown in the presence of varying concentrations of α-PLL or ε-PLL;

FIG. 6 are photomicrographs of freshly made APA microcapsules with α-PLL (left) or ε-PLL (right) at 270× magnification in an embodiment of the disclosure;

FIG. 7 are confocal scanning laser microscopy (CSLM) images of AαPA and AεPA microcapsules after 24 hour incubation in various sizes of dextran (20 kDa, 40 kDa, and 70 kDa) and the inside:outside ratio of fluorescence, in an embodiment of the disclosure;

FIG. 8 is a graph demonstrating the bactericicidal effect of α-PLL and ε-PLL on the probiotic Lactobacillus reuteri in an embodiment of the disclosure;

FIG. 9 is a graph demonstrating the mechanical stability as measured by bacterial viability of Lactobacillus reuteri NCIMB 701089 in AαPA and AεPA microcapsules over time, in an embodiment of the disclosure;

FIG. 10 is a graph demonstrating the viability of microencapsulated Lactobacillus reuteri NCIMB 701089 in AαPA and AεPA microcapsules and after secondary fermentation over time in an embodiment of the disclosure;

FIG. 11 is a graph demonstrating the bile salt hydrolase activity (measured as decreasing GDCA concentration over time) by control, AαPA microcapsules containing L. reuteri NCIMB 701089 and AεPA microcapsules containing L. reuteri NCIMB 701089 in an embodiment of the disclosure. Samples were processed after 0.5 h, 1 h, 3 h, 5 h and were analyzed with HPLC;

FIG. 12 is a graph demonstrating the bile salt hydrolase activity (measured as decreasing TDCA concentration over time) by control, AαPA microcapsules containing L. reuteri NCIMB 701089 and AεPA microcapsules containing L. reuteri NCIMB 701089 in an embodiment of the disclosure. Samples were processed after 0.5 h, 1 h, 3 h, 5 h and were analyzed with HPLC;

FIG. 13 is graph demonstrating the cell viability of L. reuteri NCIMB 701089 microencapsulated in AαPA microcapsules or AεPA microcapsules in an embodiment of the disclosure. Microcapsules were stored in 10% MRS solution at 4° C. and viability was measured every week for 2 weeks;

FIG. 14 is a graph demonstrating ferulic acid esterase activity (measured as rate of ferulic acid produced per gram microcapsules per hour) by AαPA microcapsules containing L. fermentum NCIMB 5221 and AεPA microcapsules containing L. fermentum NCIMB 5221 in an embodiment of the disclosure. Samples were processed for ferulic acid esterase activity every week for 6 weeks;

FIG. 15 is a graph demonstrating the cell viability of L. fermentum NCIMB 5221 microencapsulated in AαPA microcapsules or AεPA microcapsules in an embodiment of the disclosure. Microcapsules were stored in 10% MRS solution at 4° C. and viability was measured every week for 6 weeks;

FIG. 16 are photomicrographs of THP-1 cells immobilized in alginate beads (left) or microencapsulated in AαPA microcapsules (middle) or AεPA microcapsules (right) in an embodiment of the disclosure;

FIG. 17 is a graph demonstrating the cell viability of THP-1 cells immobilized in alginate beads or microencapsulated in AαPA microcapsules or AεPA microcapsules in an embodiment of the disclosure;

FIG. 18 are photomicrographs of activated carbon microencapsulated in AαPA microcapsules (left) or AεPA microcapsules (right) in an embodiment of the disclosure;

FIG. 19 are photomicrographs of elderberry colouring agent microencapsulated in AαPA microcapsules (left) or AεPA microcapsules (right) in an embodiment of the disclosure;

FIG. 20 are photomicrographs of beet root as an example of a food matrix microencapsulated in AαPA microcapsules (left) or AεPA microcapsules (right) in an embodiment of the disclosure;

FIG. 21 is a graph demonstrating the settling time in minutes of alginate beads (prior to alpha PLL coating), alginate beads (prior to epsilon PLL coating), AαPA microcapsules and AεPA microcapsules in 0.85% saline solution in an embodiment of the disclosure; and

FIG. 22 is a graph demonstrating the settling time of AαPA microcapsules or AεPA microcapsules in a gradient of 100%, 66.67%, 44.44%, 29.63% and 19.75% glycerol in 0.85% saline in an embodiment of the disclosure;

### DESCRIPTION

### (I) Definitions

The term “core” as used herein refers to the inner portion of the capsules of the present disclosure, wherein an active ingredient is encapsulated by a first polymer. In an embodiment, to form the core, the active ingredient is mixed with a first polymer, such as an alginate, to form a mixture. The mixture is then formed into beads optionally through the extrusion of the mixture into droplets, wherein the droplets are then contacted with solutions containing for example, optionally millimolar concentrations of calcium or barium ions (from salts such as calcium chloride or barium chloride). As a result of the calcium or barium solution, a gel-like matrix is formed around the active ingredient, which forms the core of the capsule. It will be understood by a person skilled in the art that the formation and properties of the core, and the manner in which the first polymer polymerizes or forms the core, will be dependent upon the choice of the first polymer. Accordingly, in an embodiment, some first polymers, such as alginates, use the electrostatic attractions between multivalent ions and the polymer for bonding. In another embodiment, other first polymers, such as agarose, gel at lower temperatures. In another embodiment, other first polymers, such as chitosan or silicon, covalently bond for gelation to occur to form a hydrocolloid gel. Accordingly, while first polymers such as alginate, agarose, chitosan, cellulose, gellan and kappa-carrageenan all form gel matrices, the way in which the core is formed differs. For example, kappa-carrageenan gels in a solution of potassium ions.

The term “first polymer” as used herein refers to any polymer which is able to encapsulate the active ingredient and form a gel-like matrix around the active ingredient, or throughout the entire core, allowing the active ingredient to retain its activity for any period of time. Examples of first polymers include, but are not limited to, synthetic polymers, non-biodegradable polymers, synthetic biodegradable polymers, natural biodegradable polymers, bioadhesive polymers, or naturally occurring polymers. In an embodiment, depending on the properties and characteristics of the wanted core, a person skilled in the art will be able to select a first polymer which provides those properties. For example, in an embodiment, some first polymers, such as agarose, chitosan, cellulose or silicon, will form hydrogel/hydrocolloid 3-D matrices, composed primarily of water, while other first polymers will simply form a polymeric matrix by polymerizing without the uptake of water.

The term “encapsulated” as used herein refers to the active ingredient being enclosed, or trapped, within the first polymer, wherein the first polymer either forms a gel-like matrix around the active ingredient, or the entire core is a gel-like matrix (or polymerized) core, such that the active ingredient is trapped within the polymeric matrix. In an embodiment, it will be understood that the first polymer does not form an impermeable seal around the active ingredient, but rather forms a matrix surrounding the active ingredient which allows for the passage of certain molecules in and out of the core. In another embodiment, any first polymer that has not polymerized to form part of the gel-like matrix (or polymerized core) will be present within the core of the capsule, as not all of the first polymer will form the gel-like matrix surrounding the active ingredient. In another embodiment, the entire core (all of the first polymer) of the capsule will form a gel-like matrix (fully polymerized core), such that the active ingredient is wholly contained within a polymeric or gel-like matrix. Accordingly, the core will comprise the active ingredient and a first polymer, wherein the first polymer either forms a skin of varying thickness (up to and including the entire diameter of the core), or the core is an entirely polymerized (or gel-like matrix) core, wherein the active ingredient is trapped within the polymeric matrix.

The term “active ingredient” as used herein refers to one or more compounds, such as, but not limited to, a drug, pharmaceutical, nutraceutical, biological material (e.g. a cell, such as bacteria, probiotic bacteria), inorganic material, a chemical reactant, an adhesive, a food product, a food additive, a coloring agent, an imaging agent, or a carbon nanotube. Optionally, the active ingredient has some pharmacological/biological/chemical property or other direct effect in the diagnosis, treatment, or prevention of disease, or to affect the structure or any function of the body of humans or other animals. The term includes those components that may undergo chemical change in the manufacture of the drug product and are present in the drug product in a modified form intended to furnish the specified activity or effect. There is no limitation to the active ingredient that can be used with capsules of the present disclosure.

The term “capsular wall” as used herein refers to one or more polymeric shells which surrounds the core and forms a permeable barrier, and in an embodiment, allows the passage of the active ingredient, or other substrates, nutrients, and/or waste, wherein at least one of the capsular walls comprises ε-poly-lysine or a derivative thereof. In another embodiment, the active ingredient is not able to pass through the capsular wall(s). In another embodiment, the active ingredient is not able to pass through the capsular wall(s), while other substrates of the active ingredient, nutrients and/or wastes, are able to pass through the capsular wall(s). In an embodiment, it will be understood that there are one or more, optionally two, optionally three, optionally four, optionally five or more capsular walls surrounding the core, wherein at least one of the walls is ε-poly-lysine or a derivative thereof. In another embodiment, the other capsular walls (walls which are not comprised of ε-poly-lysine), are formed of a second polymer, such as any suitable polymer for encapsulation of the core or another capsular wall, for example those polymers defined for the first polymer. In another embodiment, the capsular wall(s) are bound in any manner which provides for a structural barrier between the outside environment and the inner core, such as, but not limited to, being electrostatically bound, ionically bound or covalently bound to the core or another capsular wall. In another embodiment, the capsular wall(s) provide protection from the environment where the capsules are delivered, such as pH conditions (for example in the gastrointestinal system of an animal), enzymes (such as in the blood, organs, tissues or gastrointestinal system of an animal), immunoglobulin (such as in the blood, organs, tissues or gastrointestinal system of an animal), or the reticuloendothelial system in the cells of animals. In an embodiment, by controlling the properties and characteristics of the capsular wall(s) by the selection of an appropriate polymer for the capsular wall(s), a person skilled in the art is able to control the membrane strength, permeability selectivity and/or diffusion properties of the capsule.

The term “ε-poly-lysine or a derivative thereof” as used herein refers to a polymer comprised of repeating units of ε-lysine or any derivative thereof, wherein the repeating units of ε-lysine (A) (or a derivative (B)) have the following structure:

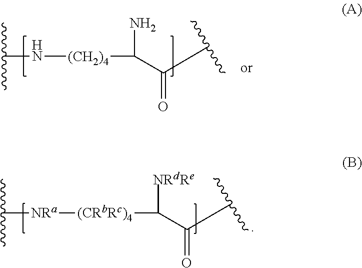

The term “derivative” as used herein in relation to the ε-poly-lysine means a

(i) synthetically modified polymer or an analogue thereof, and/or

(ii) post-translationally modified polymer, and

wherein, in one embodiment, at least one of the hydrogens of the unsubstituted ε-lysine (A) is replaced with a moiety (R^{a}-R^{e}) such as halo, (C₁-C₁₀)-alkyl, (C₁-C₁₀)-alkoxy, (C₂-C₁₀)-alkenyl, (C₂-C₁₀)-alkynyl, (C₃-C₁₀)-cycloalkyl and/or phenyl. In one embodiment, ε-poly-lysine is isolated and separated from a fermentation process (a cell source), and therefore the polymer is post-translationally modified by the organisms producing the polymer, such that, depending on the nature of the post-translational modification, R^{a}-R^{e} refer to the moieties that are attached through the post-translational modifications (for example, acyl groups, acetyl groups, formyl groups, etc).

The term “C₁₋ₙalkyl” as used herein means straight and/or branched chain, saturated alkyl groups containing from one to “n” carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the largest number of carbon atoms in the alkyl group.

The term “C₃₋ₙcycloalkyl” as used herein means a monocyclic or polycyclic saturated carbocylic group containing from three to n carbon atoms and includes (depending on the identity of n), cyclopropyl, cyclobutyl, cyclopentyl, cyclodecyl, bicyclo[2.2.2]octane, bicyclo[3.1.1]heptane and the like, where the variable n is an integer representing the largest number of carbon atoms in the cycloalkyl group.

The term “C₂₋ₙalkenyl” as used herein means straight or branched chain, unsaturated alkyl groups containing from one to n carbon atoms and one to three double bonds, and includes (depending on the identity of n) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable n is an integer representing the largest number of carbon atoms in the alkenyl group.

The term “C₂₋ₙalkynyl” as used herein means straight or branched chain, unsaturated alkyl groups containing from one to n carbon atoms and one to three triple bonds, and includes (depending on the identity of n) acetylenyl, propargyl, but-1-ynyl, but-2-ynyl, but-3-ynyl, 3-methylbut-1-ynyl, 4-methylpent-1-ynyl, penta-1,3-diynyl, hexyn-1-yl and the like, where the variable n is an integer representing the largest number of carbon atoms in the alkynyl group.

The term “halo” as used herein means a halogen atom, such as fluorine, chlorine, bromine or iodine.

The term “fluoro-substituted” with respect to any specified group as used herein means that the one or more, and optionally all, of the hydrogen atoms in the group have been replaced with a fluorine, and includes trifluoromethyl, pentafluoroethyl, fluoromethyl and the like.

### (II) Capsules

It is well known that ε-poly-L-lysine possesses high anti-microbial activity and has been shown to inhibit the growth of both gram-positive and gram-negative bacteria, including probiotic bacteria. The minimum inhibitory concentrations (MIC) for bacteria using ε-poly-L-lysine are as low as 1 μg/ml, generally below 100 μg/ml. ε-poly-L-lysine has also been shown to inhibit yeast, fungi, proteins, nucleic acid and viruses. ε-poly-L-lysine molecules are surface active agents with hydrophobic methylene groups on the inside and hydrophilic carboxyl and amino groups on the outside of the molecule in polar solutions, thus increasing its anti-microbial activity in comparison to α-poly-l-lysine.

The anti-microbial activity of ε-poly-L-lysine is based on chain length; with more than nine L-lysine residues optimal for severely inhibiting microbial growth. The chemical structure of ε-poly-L-lysine is important for its potent antimicrobial activity. The chemical modification of the α-amino groups of ε-poly-L-lysine significantly lowers the antimicrobial activity.

The proposed mechanism of the antimicrobial effect of ε-poly-L-lysine is its electrostatic absorption onto the cell surface of microorganisms, based on its cationic property. Electron microscopy has revealed this interaction to cause the stripping of the outer membrane and abnormal distribution of the cytoplasm. Differences in minimum inhibitory concentrations between bacteria, yeast, and fungi are postulated to derive from their cell surface conditions. The isoelectric point of ε-poly-L-lysine is 9.0, and as a result, the MIC is increased significantly in alkaline conditions.

However, it has now been determined that ε-poly-lysine, or a derivative thereof, is useful for encapsulating active ingredients, including bacteria, yeast, fungi, proteins, nucleic acid, viruses, without reducing or impairing the activity of the active ingredient. Without being bound by theory, it is thought that the anti-microbial activity of ε-poly-lysine, or a derivative thereof, is attenuated when incorporated into the capsules of the present disclosure as a result of the cationic moieties of ε-poly-lysine, or a derivative thereof, being complexed to the first polymer, and accordingly, bound within the capsule.

In an embodiment, cell encapsulation has been tested for a wide variety of disorders such as kidney and liver failure, gastrointestinal disorders, hypercholesterolemia, diabetes mellitus, anaemia, dwarfism, haemophilia and central nervous system insufficiencies.¹⁷⁻²⁰ Applications include transplantation, oral administration, in-vivo cell culture, reproductive technology and cytotoxicity testing.^{21,22} In addition, capsules containing hemoglobin (for use as blood substitutes), enzymes (to treat inborn errors of metabolism) or adsorbents (to treat drug overdoses) have been tested.^{21,23,24}

Encapsulation technology now ranges from macro-dimensions, to micron-dimensions and to nano-dimensions.²⁴ Macroencapsulation involves large groups of cells that are enveloped in tube or disc shape hollow devices. In comparison, microencapsulation employs a smaller cell mass as well as enzymes, peptides, drugs, vaccine or other material that is individually encased in a spherical capsule. Nanoencapsulation has been employed for blood substitutes, enzymes, peptides, drugs etc. For cell encapsulation, microcapsules are advantageous from a mass transport perspective, more difficult to mechanically disrupt and more easily implantable.²⁵

Accordingly, the present disclosure relates to a capsule comprising a core and a capsular wall comprising ε-poly-lysine or a derivative thereof. In particular, the capsule comprises;
i) a core, comprising an active ingredient encapsulated by a first polymer; and
ii) one or more capsular walls surrounding the core, wherein at least one of the capsular walls comprises a second polymer comprising s-poly-lysine or a derivative thereof.

In another embodiment, the ε-poly-lysine or derivative thereof comprises a polymer of the formula (I)

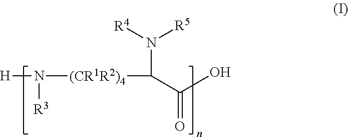

wherein

R¹ and R² are independently or simultaneously selected from H, halo, (C₁-C₁₀)-alkyl, (C₁-C₁₀)-alkoxy, (C₂-C₁₀)-alkenyl, (C₂-C₁₀)-alkynyl, (C₃-C₁₀)-cycloalkyl and phenyl, wherein the latter six groups are optionally substituted,

R³ is selected from H, (C₁-C₆)-alkyl, (C₂-C₆)-alkenyl and (C₂-C₆)-alkynyl, wherein the latter three groups are optionally substituted,

R⁴ and R⁵ are independently selected from H, (C₁-C₁₀)-alkyl, (C₂-C₁₀)-alkenyl, (C₂-C₁₀)-alkynyl, (C₃-C₁₀)-cycloalkyl and phenyl, wherein the latter six groups are optionally substituted,

n is an integer from 1 to 100,

the optional substituents are selected from one to five of halo, C₁₋₆alkyl and fluorosubstituted

and all stereoisomers and enantiomers thereof.

In another embodiment, R¹ and R² are independently or simultaneously selected from H, halo, (C₁-C₆)-alkyl, (C₁-C₆)-alkoxy, (C₂-C₆)-alkenyl, (C₂-C₆)-alkynyl, (C₃-C₆)-cycloalkyl and phenyl, wherein the latter six groups are optionally substituted. In a further embodiment, R¹ and R² are independently or simultaneously selected from H and optionally substituted (C₁-C₄)-alkyl. In another embodiment, R¹ and R² are independently or simultaneously H, optionally substituted methyl or optionally substituted ethyl.

In another embodiment of the disclosure, R³ is selected from H and optionally substituted (C₁-C₆)-alkyl. In another embodiment, R³ is selected from H and optionally substituted (C₁-C₄)-alkyl. In another embodiment, R³ is selected from H, optionally substituted methyl and optionally substituted ethyl. In another embodiment, R³ is H.

In another embodiment of the disclosure, R⁴ and R⁵ are independently or simultaneously selected from H, (C₁-C₆)-alkyl, (C₂-C₆)-alkenyl, (C₂-C₆)-alkynyl, (C₃-C₆)-cycloalkyl and phenyl, wherein the latter six groups are optionally substituted. In a further embodiment, R⁴ and R⁵ are independently or simultaneously selected from H and optionally substituted (C₁-C₄)-alkyl. In a further embodiment, R⁴ and R⁵ are independently or simultaneously H, optionally substituted methyl or optionally substituted ethyl.

In another embodiment, n is an integer from 1 to 50, optionally, 10 to 50, optionally 15 to 45, optionally 20 to 40, optionally from 25 to 35, optionally 25 to 30.

In another embodiment of the disclosure, the polymer comprising ε-poly-lysine or a derivative thereof is a polymer of the formula

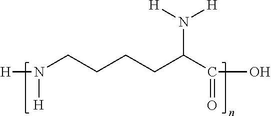

wherein n is an integer from 10 to 50, optionally 15 to 45, optionally 20 to 40, optionally from 25 to 35, optionally 25 to 30.

In a further embodiment of the disclosure, wherein the polymer comprising ε-poly-lysine or a derivative thereof is a polymer of the formula

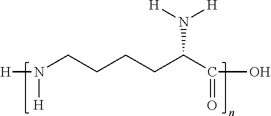

wherein n is an integer from 10 to 50, optionally 15 to 45, optionally 20 to 40, optionally from 25 to 35, optionally 25 to 30.

In another embodiment of the disclosure, the ε-poly-lysine or derivative thereof is isolated from a product of yeast or bacterial fermentation. In another embodiment, when the ε-poly-lysine or derivative is isolated from a product of yeast or bacterial fermentation, the microorganism post-translationally modifies the ε-poly-lysine into a ε-poly-lysine derivative. Examples of post-translational modifications include, but are not limited to, acylation (for example, O-acylation, S-acylation, or N-acylation); acetylation; formylation; lipoylation (for example, the attachment of a lipoate (e.g. C₈) functional group); myristoylation (attachment of myristate, a C₁₄ saturated acid); palmitoylation (attachment of palmitate, a C₁₆ saturated acid); alkylation (for example, the addition of an alkyl group, e.g. methyl, ethyl); methylation; isoprenylation or prenylation, the addition of an isoprenoid group (for example, farnesol and geranylgeraniol); amidation at C-terminus; amino acid addition (for example, arginylation, polyglutamylation); polyglycylation; diphthamide formation; gamma-carboxylation; glycosylation; polysialylation (addition of polysialic acid); glypiation (glycosylphosphatidylinositol (GPI) anchor formation); attachment of a heme moiety; hydroxylation; hypusine formation; iodination; covalent attachment of nucleotides or derivatives; adenylation; ADP-ribosylation; flavin attachment; nitrosylation; S-glutathionylation; oxidation; phosphopantetheinylation (for example, the addition of a 4′-phosphopantetheinyl moiety); phosphorylation; pyroglutamate formation; sulfation; selenoylation (co-translational incorporation of selenium in selenoproteins); glycation (the addition of a sugar molecule to a protein without the controlling action of an enzyme); biotinylation; acylation and pegylation.

The present disclosure also encompasses all isomers of polymers of formula (I) and their pharmaceutically acceptable derivatives, including all geometric, tautomeric and optical forms, and mixtures thereof (e.g. racemic mixtures). Where additional chiral centres are present in polymers of formula (I), the present disclosure includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

In an embodiment, capsules comprising epsilon-poly-D-lysine, and derivatives thereof, are used for non-oral applications (applications where the capsules are administered within the body of an animal), such as for fermentation, topical use, biomass production/optimization.

In another embodiment of the disclosure, the first polymer is a synthetic polymer, a non-biodegradable polymer, a synthetic biodegradable, polymer a natural biodegradable polymer, a bioadhesive polymer, or a naturally occurring polymer.

In another embodiment, the synthetic polymer is selected from polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellullose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride, polystyrene and polyvinylpyrrolidone, copolymers and mixtures thereof.

In another embodiment, the non-biodegradable polymer is selected from ethylene vinyl acetate, poly(meth)acrylic acid, polyamides, copolymers and mixtures thereof.

In a further embodiment, the synthetic biodegradable polymer is selected from lactic acid, glycolic acid, polyanhydrides, poly(ortho)esters, polyurethanes, poly(butic acid), poly(valeric acid), poly (lactide-co-caprolactone), copolymers and mixtures thereof.

In another embodiment of the disclosure, the natural biodegradable polymer is selected from polysaccharides, alginate and other polysaccharides including dextran and cellulose, collagen, chemical derivatives thereof, albumin and other hydrophilic proteins, zein and other prolamines and hydrophobic proteins, copolymers and mixtures thereof.

In an embodiment of the disclosure, the bioadhesive polymer is selected from bioerodible hydrogels, polyhyaluronic acids, casein, gelatin, glutin, polyanhydrides, polyacrylic acid, alginate, chitosan, poly(methyl methacrylates), poly(ethyl methacrylates), poly(butylmethacrylate), poly (isobutyl methacrylate), poly (hexylmethacrylate), poly (isodecyl methacrylate), poly (lauryl methacrylate), poly (phenyl methacrylate), poly(methyl acrylate), poly(isopropyl acrylate), poly(isobutyl acrylate), and poly(octadecyl acrylate), copolymers and mixtures thereof.

In a further embodiment, the naturally occurring polymers are selected from alginate, chitosan, cellulose, pectin, gelatine, genepin, and agarose.

In an embodiment, the generation of the core is obtained by using any of the above polymers, or polymeric monomer units to form the polymer, and a person skilled in the art would know and understand the necessary conditions for the formation of the core using any particular first polymer. For example, when alginate is the first polymer, the cationic gelation of the alginate is governed by diffusion, and gelation (or polymerization) of the alginate occurs as calcium ions diffuse through an alginate droplet (core). Accordingly, for alginate, the gelation or polymerization occurs first on the outside of the droplet or core, and continue through to the inner core as the calcium diffuses through. For other polymers, mild chemical polymerization reactions occur to form the core.

In another embodiment, the first polymer is alginate and the polymer of the formula (I) is epsilon-poly-L-lysine.

In another embodiment of the disclosure, the capsular wall(s) which are not comprised of a polymer of the formula (I), comprise polymers which are suitable for the encapsulation of the core or other capsular walls, and are optionally defined as for the first polymer, such as a synthetic polymer as described above, a non-biodegradable polymer as described above, a synthetic biodegradable polymer as described above, a natural biodegradable polymer as described above, a bioadhesive polymer as described above, or a naturally occurring polymer as described above.

In another embodiment of the disclosure, the capsule comprises a capsule selected from the following:

| | | 1^{st} | 2^{nd} | 3^{rd} | 4^{th} |
|---|---|---|---|---|---|
| | First | capsular | Capsular | Capsular | Capsular |
| Capsule | Polymer | wall | Wall | Wall | Wall |
| 1 | Alginate | ε-poly-l- | Alginate | — | — |
| | | lysine | | | |
| 2 | Alginate | ε-poly-l- | Pectin | ε-poly-l- | Alginate |
| | | lysine | | lysine | |
| 3 | Alginate | Chitosan | Alginate | ε-poly-l- | — |
| | | | | lysine | |
| 4 | Alginate | Chitosan | Alginate | ε-poly-l- | Alginate |
| | | | | lysine | |

In another embodiment, the first polymer is alginate and the polymer of the formula (I) is epsilon-poly-L-lysine. In another embodiment, a capsule comprising an alginate core, a first capsular wall comprising epsilon-poly-L-lysine, and a second capsular wall comprising alginate, possesses a capsular wall that is at least two times, optionally three times, optionally four times thicker than that of a capsule comprising an alginate core, a first capsular wall comprising alpha-poly-L-lysine and a second capsular wall comprising alginate.

In another embodiment, a capsule according to the present disclosure has a porosity which allows at least 20%, optionally at least 30%, optionally at least 34% of a 40 kDa dextran. In another embodiment, a capsule comprising an alginate core, a first capsular wall comprising epsilon-poly-L-lysine, and a second capsular wall comprising alginate, possesses a porosity which allows at least 20%, optionally at least 30%, optionally at least 34% of a 40 kDa dextran.

In another embodiment of the disclosure, a capsule according to the present disclosure has a mechanical stability such that at least 100, optionally 500, and optionally 1.00 cfu/g (colony forming units/gram) of a probiotic bacteria remain viable after shaking 2.5 g of capsules (containing bacteria) in 10 mL of 10% MRS at 37° C. for 24 hours. In another embodiment, a capsule comprising an alginate core, a first capsular wall comprising epsilon-poly-L-lysine, and a second capsular wall comprising alginate, has a mechanical stability such that at least 100, optionally 500, and optionally 1.00 cfu/g (colony forming units/gram) of a probiotic bacteria remain viable after shaking 2.5 g of capsules (containing bacteria) in 10 mL of 10% MRS at 37° C. for 24 hours.

In another embodiment, the active ingredient is a drug, biological material, inorganic material, a chemical reactant, an adhesive, a food product, a food additive, a coloring agent or an imaging agent.

In another embodiment of the disclosure, the inorganic material is a mineral, a vitamin, food colouring or activated carbon.

In a further embodiment, the biological material is a live cell, a cell isolate, a cell component, an enzyme, a protein, an immunoglobulin, or nucleic acid. The live cell is optionally an archea, a bacteria, a fungi, a plant cell, or an animal cell. The bacteria are typically a probiotic microorganism or fermentation organism of the genus Lactobacillus, Bifidobacteria, Pediococcus, Streptococcus, Enterococcus, or Leuconostoc. The live cell optionally expresses an enzyme selected from bile salt hydrolase (BSH), ferulic acid esterase (FAE), and nitrate reductase (NiR). The fungi is readily selected from species such as Torula species, baker's yeast, brewer's yeast, a Saccharomyces species, optionally S. cerevisiae, a Schizosaccharomyces species, a Pichia species optionally Pichia pastoris, a Candida species, a Hansenula species, optionally Hansenula polymorpha, and a Klyuveromyces species, optionally Klyuveromyces lactis. The animal cell is optionally a beta islet cell. The nucleic acid is typically selected from RNA, antisense RNA, siRNA, plasmid, cosmid, dsDNA, or ssDNA. The biological material is optionally selected from a virus, an attenuated virus, and material intended for the purpose of vaccination.

In another embodiment, the food product is selected from an omega oil and a fish oil.

In another embodiment of the disclosure, the capsule is a nanocapsule between 1 nm and less than 1000 nm. In another embodiment, the capsule is a microcapsule between greater than 1 μm and less than 1000 μm. In a further embodiment, the capsule is a macrocapsule greater than 1000 μm.

In another embodiment, the capsules of the disclosure have an improved buoyancy, or a decreased tendency, to settle in a liquid as compared to other capsules which are not comprised of ε-poly-l-lysine (such as a capsule comprised of α-poly-l-lysine. In one embodiment, a capsule of the disclosure has a settling time of at least about 2.5 minutes in a 0.85% saline solution, or at least about 3 minutes, or at least about 3.5 minutes.

In a further embodiment of the disclosure, the capsule further comprises pharmaceutically acceptable excipients. In another embodiment, the pharmaceutically acceptable excipients comprise a hollow fiber, cellulose nitrate, polyamide, lipid-complexed polymer, a lipid vesicle a siliceous encapsulate, cellulose sulphate/sodium alginate/polymethylene-co-guanidine (CS/A/PMCG), cellulose acetate phthalate, calcium alginate, k-carrageenan-Locust bean gum gel, gellan-xanthan, poly(lactide-co-glycolides), carageenan, starch polyanhydrides, starch polymethacrylates, polyamino acids or enteric coating polymers.

In another embodiment, the capsules of the present disclosure are suitable for oral administration, topical administration, transplantable, suitable for use in as an ex-vivo device or suitable for use in a fermentation process.

In an embodiment, the capsules of the present disclosure are administered in a standard manner for the treatment of diseases, for example orally, parenterally, sub-lingually, dermally, intranasally, transdermally, rectally, via inhalation or via buccal administration.

In another embodiment, the capsules of the present disclosure when given orally are formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the capsules are in the form of a tablet, any pharmaceutical carrier routinely used for preparing solid formulations may be used. Examples of such carriers include magnesium stearate, terra alba, talc, gelatin, acacia, stearic acid, starch, lactose and sucrose.

In an embodiment, the capsules of the present disclosure are delivered by ingestion, subcutaneous injection, intravenous injection, inhalation, intraocular/periocular injection, or nasal inhalation,

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of formula (I) or a pharmaceutically acceptable derivative thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Numerous techniques for microencapsulation, both physical and chemical, are available depending on the nature of the encapsulated substance or encapsulate and on the type of encapsulating material. Physical methods of encapsulation include, for example, spray drying, spray chilling, rotary or spinning disk atomization, fluid bed coating, pan coating, stationary nozzle co-extrusion, centrifugal head co-extrusion and submerged nozzle co-extrusion. Spray drying involves the dispersion of the encapsulate into a concentrated solution of coating material. The resultant emulsion is then atomized into a spray of droplets. Spray-chilling involves mixing cool particles of the encapsulate with hot-coating materials to create either a solution or dispersion. This mixture is then atomized into a chamber, where it is contacted with a cool air stream that causes the atomized droplets to solidify, forming the encapsulated product. In rotary of spinning disk atomization, the encapsulate is dispersed into the coating material and the mixture is advanced onto a turning disk wherein droplets are then thrown off of the rim of the disk resulting in discrete particle formation. In fluid bed coating, particles to be encapsulated are suspended on a jet of air and then covered by a spray of liquid coating material. The capsules are then solidified by cooling or solvent vaporization. In pan coating, solid particles are mixed with a dry coating material and the temperature is raised so that the coating material melts and encloses the core particles, and then is solidified by cooling. In co-extrusion processes, such as stationary nozzle, centrifugal head or submerged nozzle, the encapsulate and coating material are mixed together and the resulting suspension is pushed through a nozzle. This movement forms an unbroken rope which naturally splits into round droplets directly after clearing the nozzle. The continuous walls of these droplets are solidified either by cooling or gelation.

Chemical methods of encapsulation include, for example, interfacial polymerization, coacervation or phase separation, solvent evaporation or extraction, matrix polymerization, in-situ polymerization, liposome technology and nanoencapsulation. Interfacial polymerization is characterized by membrane formation via the rapid polymerization of monomers at the surface of the droplets or particles of dispersed core material. A multifunctional monomer is dissolved in the core material, and this solution is dispersed in an aqueous phase. In situ polymerization is very similar to interfacial polymerization with the distinguishing characteristic that no reactants are included in the core material. Microencapsulation by coacervation or phase separation involves three main steps: phase separation of the coating polymer solution, adsorption of the coacervate around the encapsulate and solidification of the microparticles. Microcapsule preparation by solvent extraction/evaporation consists of the dissolution or dispersion of the encapsulate often in an organic solvent containing the matrix forming material, the emulsification of this organic phase in a second continuous, frequently aqueous, phase immiscible with the first one and the extraction/evaporation of the solvent from the dispersed phase by the continuous phase resulting in solid microcapsules. Matrix polymeration occurs when core material is imbedded in a polymeric matrix during formation of the particles. Liposome technology involves a region of aqueous solution inside a hydrophobic membrane wherein dissolved hydrophilic solutes cannot readily pass through the lipid layer. Nanoencapsulation has evolved from and can be considered to be the miniaturisation of microencapsulation.

In another embodiment of the disclosure, there is also a method for the production of a capsule comprising i) a core, comprising an active ingredient encapsulated by a first polymer; and ii) one or more capsular walls surrounding the core, wherein at least one of the capsular walls comprises ε-poly-lysine or a derivative thereof as defined above, where the method includes contacting the active ingredient with the first polymer to form the core, in which a gel-like matrix is formed around the active ingredient, and wherein the core is then encapsulated by one or more capsular walls by contacting the core with ε-poly-lysine or a derivative thereof or any other polymer suitable for encapsulation. When there is more than one capsular wall, the process is repeated by further contact with ε-poly-lysine or a derivative thereof or any other polymer suitable for encapsulation.

### (III) Uses

Bacteriophage attack constitutes a major problem in the dairy industry resulting in infected batches and inefficient production. The fermentation and fermented food industry has used several techniques to fight phage attack including the use of starters containing phage-unrelated or phage-insensitive strains, production of phage-free bulk starter with septic propagation systems, phage-inhibitory media, segregation of starter room and process equipment, removal of deposits on bulk starter vessels, and ensuring the head space is minimised in bulk starter vessels and sterilised. Also, these industries have tried to limit the effect of phages in starter cultures including minimising the concentration of phage in processing plants, rotating cultures, using air conditioning, using aerosol generation/fumigation, cleaning/chlorination of vats between refills, and good factory design.

Another approach to protecting culture from phage attack is to encapsulate the culture and physically protect the organisms from potential phage attack. The use of immobilization to protect culture is further benefited in continuous fermentation, where the possibility of phage contamination would otherwise increase. Bacteria released into the fermentation medium and bacteria on the surface of beads are not resistant to attack; thus, phages still have the ability to multiply in an immobilized culture system, even though cells inside the bead are protected.

Accordingly, in one embodiment, microencapsulation of fermentation bacteria utilizing capsules comprising ε-polylysine or a derivative thereof protects the culture from bacteriophage attack during fermentation and prevents the infection of starter cultures used for the production of fermented food products such as yogurt, cheese, beer, wine, tofu, etc. reducing the number of infected batches and improving culture production efficiency. In one embodiment, there is included a method for preventing, or reducing, phage attack of a fermentation organism during a fermentation process, wherein the fermentation process comprises:

(i) contacting a substrate with a capsule as defined in herein, wherein the active ingredient comprises a fermentation organism, and

(ii) fermenting the substrate.

In another embodiment, the fermentation organisms comprise mammalian, bacterial, yeast, or insect cells. In another embodiment, the bacterial or yeast cells comprise a starter culture for the production of a fermented food product. In a further embodiment, the fermentation organisms are protected from viral or phage attack. In another embodiment, the capsules comprising the fermentation organisms are also protected from phage attack before, during or after a fermentation process. Accordingly, in one embodiment, capsules of the disclosure comprising fermentation organisms can be prepared according to the present disclosure and stored for a period of time before being used in a fermentation process, as the capsules prevent or reduce the opportunity of phage attack.

In another embodiment, a capsule of the present disclosure, such as an alginate-ε-poly-L-lysine-alginate microcapsule, containing biologic material such as RNA, antisense RNA, siRNA, plasmid, cosmid, dsDNA, or ssDNA is used for delivery of nucleic acids for gene delivery by ingestion, transplantation, gene gun delivery, microinjection, intravenous injection, inhalation, intraocular/periocular injection, nasal inhalation, etc.

In another embodiment, a capsule of the present disclosure, such as an alginate-ε-poly-L-lysine-alginate microcapsule, containing fungi or yeast is used for the fermentation of products from substrate in the appropriate growth media and under the correct growth conditions. In an embodiment, the encapsulation of yeast that are genetically engineered to produce therapeutics such as insulin or tissue plasminiogen activator (tPA) is an effective way to produce and harvest complex biotherapeutic agents.

In another embodiment, a capsule of the present disclosure, such as an alginate-ε-poly-L-lysine-alginate microcapsule, containing immunoglobulins is used for the treatment of various systemic, topical, or intra-gastrointestinal luminary disease. In another embodiment, immunoglobulins in the form of targeted monoclonal antibody therapy are employed to treat diseases such as rheumatoid arthritis, multiple sclerosis, psoriasis, and many forms of cancer including non-Hodgkin's lymphoma, colorectal cancer, head and neck cancer and breast cancer.

In another embodiment, a capsule of the present disclosure, such as an alginate-ε-poly-L-lysine-alginate microcapsule, is constructed containing a drug. In an embodiment, the capsule protects the drug from the harsh environment of the upper gastrointestinal tract and ensures greater delivery of active drug ingredients. In another embodiment, the capsule provides targeted delivery of drugs to different parts of the gastrointestinal tract. For example, the delivery of thalidomide to the illeocecal junction of the gastrointestinal tract for the treatment of Crohn's disease and ulcerative colitis is accomplished using a capsule of the present disclosure.

In another embodiment, a capsule of the present disclosure, such as a alginate-ε-poly-L-lysine-alginate microcapsule, containing minerals, vitamins, or cofactors is delivered orally to aid in the effective delivery of active inorganics.

In another embodiment, a capsule of the present disclosure, such as a alginate-ε-poly-L-lysine-alginate microcapsule, containing a virus, attenuated virus, heat killed virus, etc is used for the purpose of vaccination. In an embodiment, the capsules are delivered by ingestion, subcutaneous injection, intravenous injection, inhalation, intraocular/periocular injection, or nasal inhalation, such that the viral antigen is delivered to the immune system and an immunological response is raised.

In another embodiment, a capsule of the present disclosure, such as a alginate-ε-poly-L-lysine-alginate microcapsule, containing microorganisms is used in a process of continuous fermentation. In an embodiment, there is included a method for the fermentation of a substrate, comprising contacting the substrate with a capsule of the disclosure, wherein the capsule comprises microorganisms which ferment the substrate. In one embodiment, the substrate is for example a dairy substrate, in which fermentation organisms are involved in the preparation of dairy products, such as cheese, yoghurt, milk etc. In an embodiment, microencapsulated or immobilized bacteria, yeast, insect, or mammalian cells are packed into a column and placed into a fermenter with controlled temperature, pH conditions, stirring, antifoam, etc. Growth media and produced gas can be drawn from the fermenter and new media are added in replacement, which sets up a continuous flow with a rate that is dependent on the rate of media exchange. The rate will depend on the use of nutrients, production of toxic wastes (to the fermenting cells), and whether there is the correct quantity of desired product formation. One example would be microencapsulated yeast for the continuous fermentation of alcohol.

In another embodiment, the active ingredient is a carbon nanotube which carries a drug, pharmaceutical, therapeutic molecule or nutraceutical, or any other active ingredient defined herein, and acts as a drug delivery device to a targeted area, such as a cell, tissue or organ.

It will be understood by those skilled in the art that by the selection of an appropriate first polymer, active ingredient and the number and identity of capsular walls, a capsule will be obtained having suitable properties and characteristics for the particular purpose of the capsule, such as permeability, capsule stability, etc. For example, when the active ingredient is insulin producing beta islet cells, insulin is produced within the capsule and diffuses out through the capsular walls to affect its activity. In another example, when the bile salt hydrolysis (BSH) active bacteria Lactobacillus reuteri is the active ingredient, BSH enzyme is produced by the bacteria and maintained within the capsule. Accordingly, bile acids are able to diffuse through the capsular walls into the core of the capsule and are deconjugated by the enzyme, wherein the product is precipitated within the capsule.

The present disclosure also includes a method for delivering an active ingredient to the gastrointestinal system of an animal comprising orally delivering a capsule according to the present disclosure to the animal.

In another embodiment, there is also included a method for delivering probiotic organisms to the gastrointestinal system of an animal comprising orally delivering a capsule containing probiotic organism(s) according to the present disclosure to the animal. In another embodiment, the probiotic organism is protected from acidic pH, IgA, enzymatic attack, bacteriosins, and bacteriophage attack as a result of the probiotic organisms being surrounded by the capsule as defined herein.

In another embodiment, there is also included a method for transplanting eukaryotic cells into an animal comprising transplanting a capsule according to the present disclosure into the animal. In an embodiment, the capsule is transplanted into the animal by surgically implanting the capsule(s) into the animal, for example, into the peritoneum through an injection using a needle. In another embodiment, the capsule(s) are transplanted by implanting into the dermis of the skin or implanted intra-ocularly.

In another embodiment, there is also included a method for delivering vectors to the gastrointestinal system of an animal comprising orally delivering a capsule according to the present disclosure into the gastrointestinal system. In an embodiment, examples of vectors include virus, plasmids, cosmids, etc., which act as delivery vehicles (vectors) of DNA or RNA, or any nucleic acid, into an animal.

In another embodiment, there is also included a method for haemoperfusion of an animal comprising delivering a haemoperfusion device comprising a capsule according to the present disclosure. In an embodiment, a capsule comprising a sorbent, such as activated carbon, is optionally lyophilized and placed within the haemoperfusion device, wherein blood runs through the device and back into the patient, and the organic toxic substances are absorbed by the sorbent. In an embodiment, when used with a hemodialysis device there is equilibration between the blood and hemodyalysate as well.

In another embodiment, there is also included a method for fermentation of microorganisms wherein the fermentation is by a capsule according to the present disclosure. In an embodiment, capsules comprising bacteria or yeast are packed into a column and placed into a fermenter with controlled temperature, pH conditions, stirring, antifoam, etc. Growth media and produced gas are drawn from the fermenter and new media are added in replacement, which sets up a continuous flow with a rate that is dependent on the rate of media exchange. The rate will depend on the use of nutrients, production of toxic wastes (to the fermenting cells), and whether there is the correct quantity of desired product formation. In embodiment, the method of fermentation is useful for ethanol fermentation.

The present disclosure also includes a use of a capsule according to the present disclosure for delivering an active ingredient to the gastrointestinal system of an animal comprising orally delivering a capsule of the present disclosure to the animal.

In another embodiment, there is also included a use of a capsule according to the present disclosure for the delivery of probiotic organisms to the gastrointestinal system of an animal comprising orally delivering a capsule containing probiotic organism(s) according to the present disclosure to the animal.

In another embodiment, there is also provided a use of a capsule of the present disclosure for transplanting eukaryotic cells into an animal comprising transplanting a capsule according to the present disclosure into the animal.

In another, there is also included a use of a capsule of the present disclosure for delivering vectors into the gastrointestinal system of an animal comprising orally delivering a capsule containing the vectors according to the present disclosure into the gastrointestinal system.

In another embodiment, there is also included a use of a capsule of the present disclosure for the haemoperfusion of an animal wherein the haemoperfusion device contains a capsule according to the present disclosure. In an embodiment, a capsule comprising a sorbent, such as activated carbon, is optionally lyophilized and placed within the haemoperfusion device, wherein blood runs through the device and back into the patient, and the organic toxic substances are absorbed by the sorbent. In an embodiment, when used with a hemodialysis device there is equilibration between the blood and hemodyalysate as well.

In another embodiment, there is also included a use of a capsule of the present disclosure for the fermentation of microorganisms wherein the fermentation is performed by a capsule according to the present disclosure. In an embodiment, capsules comprising bacteria or yeast are packed into a column and placed into a fermenter with controlled temperature, pH conditions, stirring, antifoam, etc. Growth media and produced gas are drawn from the fermenter and new media are added in replacement, which sets up a continuous flow with a rate that is dependent on the rate of media exchange. The rate will depend on the use of nutrients, production of toxic wastes (to the fermenting cells), and whether there is the correct quantity of desired product formation. In embodiment, the method of fermentation is useful for ethanol fermentation.

The following non-limiting examples are illustrative of the present disclosure:

### EXAMPLES

### Materials and Methods (General)

Bacterial seeding and growth: The surfaces of frozen glycerol bacterial stocks were scratched with a sterile wooden stick to streak MRS agar plates. After an overnight incubation at 37° C. under anaerobic conditions, a single colony was picked with a metallic loop under sterile conditions and transferred into a tube containing 10 mL of MRS. The cultures were incubated overnight at 37° C. for experimental use.

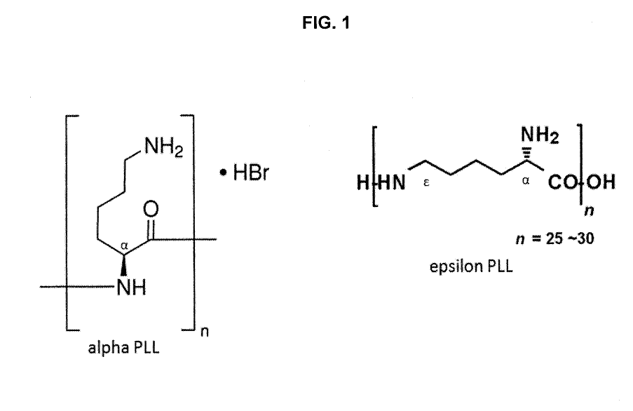

Microencapsulation: Microcapsules were prepared with an 8% cell load and a 1.75% alginate concentration using a 200 μm sized nozzle. The coating process was the following: first, alginate beads were drained of CaCl₂, second, alginate beads were washed in 0.85% (w/v) NaCl for 10 minutes; third, alginate beads were coated in 0.1% (w/v) α-PLL or ε-PLL (structure shown in FIG. 1) for 20 minutes; fourth, alginate-PLL microcapsules were washed with 0.85% (w/v) NaCl for 10 minutes; fifth, alginate-PLL microcapsules were coated with 0.1% (w/v) alginate for 20 minutes; and finally the alginate-PLL-alginate microcapsules were washed with 0.85% (w/v) NaCl for 10 minutes.

### Example 1

### Morphology and Structural Properties of AεPA Versus AαPA Microcapsules

Preparation of APA microcapsules for transmission electron microscopy (TEM):

To prepare and fix the AαPA or AεPA microcapsules in preparation for TEM, microcapsules were washed (×3) with buffer for 30 min., post-fixed with 1% aqueous OsO₄, and 1.5% aqueous potassium ferrocyanide for 2 h at 4° C. The samples were then washed (3×) with ddH₂O for 15 min., dehydrated with acetone: 30%, 50%, 70%, 80%, 90%, (3×) 100% each for 15 min. and infiltrated with epon/acetone 1:1 overnight, 2:1 all day, 3:1 overnight, pure epon the following day for day 4 h. Samples were then embedded with a change of fresh epon., polymerization in 58° C. oven for 4 h. The samples were trimmed and cut into 90-100 nm thick sections, put onto a 200 mesh copper grid, sections on grids were stained with uranyl acetate for 6 minutes and then Reynold's lead for 5 min.

### Specimen Preparation for SEM:

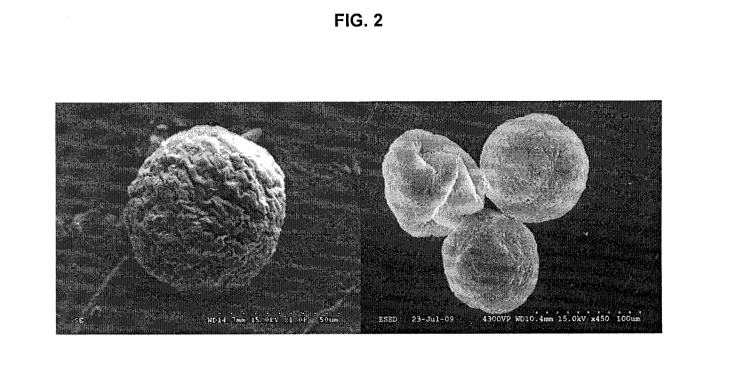

Samples previously stored in 0.85% saline were fixed overnight in 4° C. in 2.5% glutaraldehyde at 4° C., microcapsules were transferred to amyl acetate solutions of increasing concentration (by serials of 25:75, 50:50, 75:25, 100:0 for amyl acetate:ethanol, v/v) for critical point drying, samples were desiccated by critical point drying and mounted onto a solid support coated with a PLL film for imaging, images taken with a Hitachi S-3000N Variable Pressure-SEM (VP-SEM) w/Oxford INCA EDS and HKL EBSD and are shown in the micrograph on the left in FIG. 2.

### Specimen Preparation for SEM (Dehydration of Capsules by Gradient Ethanol):

10-15 microcapsules were placed into a labeled glass vial, residual liquid was discarded and 30 mL 30% ethanol was added, the contents were mixed by gentle rotation at RT for 15 min., the ethanol in the vial was discarded and replaced with 30 mL new ethanol of increasing concentration (e.g., 50%), steps 3 and then step 4 were repeated using a gradient of increasing ethanol concentration, e.g., 70%, 80% and 90%, finally the microcapsules were suspended in a solution of 100% ethanol prior to performing a medium transfer from ethanol to amyl acetate (by serials of 25:75, 50:50, 75:25, 100:0 for amyl acetate:ethanol, v/v) for critical point drying (Emitech K850), the samples were then subjected to critical point drying under CO₂ then mounted onto a sPLL coated glass support, images taken with HITACHI S-4300SE/N (VP-SEM) with ESED detection and are shown in the micrograph on the right in FIG. 2.

### Confocal Scanning Laser Microscopy (CSLM):

Empty microcapsules were prepared with a final concentration of 1.75% alginate using a 200 μm sized nozzle. The coating process was the same as described in Microscopy. Microcapsules were left in 0.85% saline overnight at room temperature, then drained and ca. 150 microcapsules were transferred in 150 μl of different fluorescent dextran solutions (1 mg/ml). The different solutions contained either 20 kDa, 40 kDa, or 70 kDa FITC-Dextran. The dextran solutions with the microcapsules were stored in the dark and at room temperature for 24 h before CSLM analysis.

### Mechanical Stability:

To assay the physical strength of the APA ε-PLL coated microcapsules versus the α-PLL, a mechanical stability test was designed. 2.5 g of microcapsules (α/ε-PLL) were added to 10 mL of 10% MRS in 50 mL falcon tubes. All tubes were stored in a 37° C. incubator for 72 h and exposed to shaking at 150 rpm or no shaking. At each time point, microcapsules were removed, filtered and rinsed with 0.85% (w/v) NaCl. Microcapsules were transferred to 2 mL Eppendorf tubes and 0.1M sodium citrate was added (1:20 dilution). Tubes were vortexed until encased bacteria were released. Serial dilutions were performed and cell viability was assessed.

### Discussion

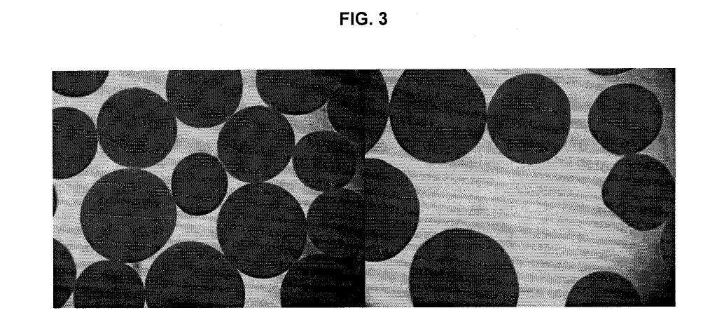

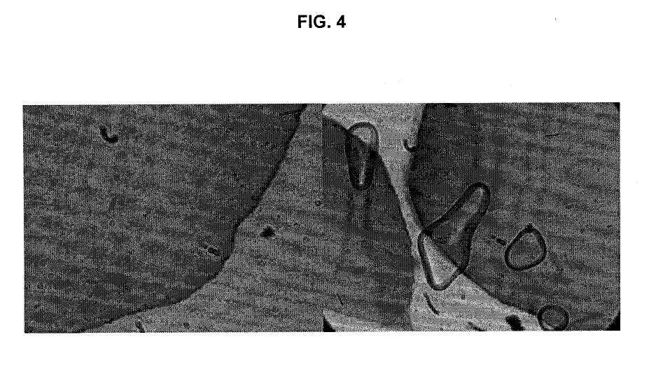

To grossly compare the morphology and membrane structure of alginate-alpha-polylysine-alginate (AαPA) and alginate-epsilon-polylysine-alginate (AεPA) microcapsules, light photomicrographs at 270× magnification (see FIG. 3: α-PLL, left and ε-PLL, right) and 1000× magnification oil emersion photomicrographs were taken (see FIG. 4: α-PLL, left and ε-PLL, right). Light photomicrographs of AαPA and AεPA microcapsules showed round looking microspheres that have a regular shape, similar spherical morphology, and no significant difference between AαPA and AεPA microcapsules. Largely the differences between membrane characteristics were indistinguishable by light microscopy at 270× magnification. The toluidine blue stained microcapsules viewed in oil emersion, however, showed that AεPA have a thicker membrane than do AαPA microcapsules.

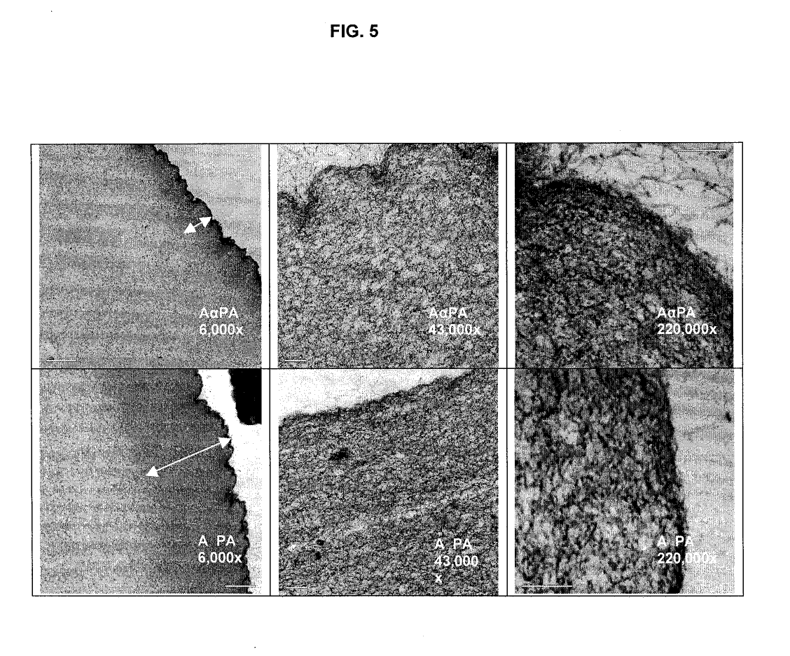

To compare the membrane structural characteristics, transition electron photomicrographs were taken of alginate-alpha-polylysine-alginate (AαPA) and alginate-epsilon-polylysine-alginate (AεPA) microcapsule membranes and are shown in FIG. 5. The top row shows the AαPA microcapsule membrane at ascending magnification from left to right (6,000×, 43,000×, and 220,000×). The bottom row shows the AεPA microcapsule membrane at ascending magnification from left to right (6,000×, 43,000×, and 220,000×). The reference line in each measure 2 μm, 0.2 μm, and 100 nm from left to right respectively. The membrane of the AεPA microcapsules, as measured by the region of increased density, is greater than 3× the thickness of that of AαPA microcapsules. This indicates that there may be a difference between AαPA and AεPA, in terms of membrane assembly and structure, which may be indicative of a difference in membrane structural integrity, electrostatic properties, mass transfer properties, biocompatibility, etc. Without being bound by theory, the more linear structure of ε-polylysine results in deeper penetration of the polyamino acid and a thicker membrane with different mass transfer properties, whereas the more globular structure of α-polylysine results in a more superficial interaction with alginate and a thinner membrane.

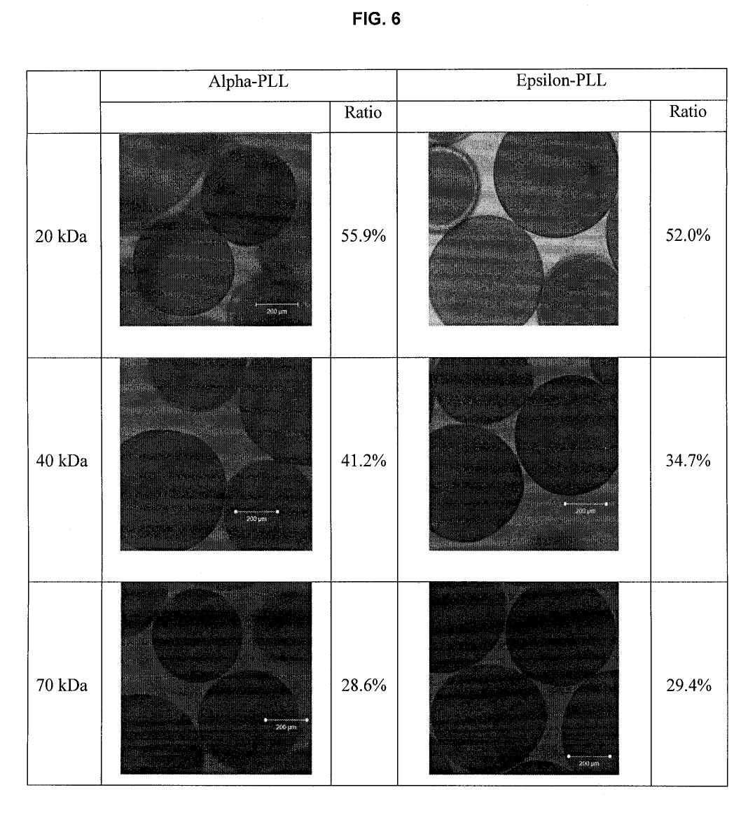

To compare the relative porosity of AαPA and AεPA microcapsule membranes, confocal scanning laser microscopy (CSLM) images of AαPA and AεPA microcapsules were taken after 24 hours incubation in various sizes of dextran (20 kDa, 40 kDa, and 70 kDa). The images provide a measure of fluorescence inside and outside of the capsules. The inside:outside ratio of fluorescence was determined and indicates the relative quantity of FITC-dextran that migrated into each microcapsule during the incubation period. The calculated ratios for 20 kDa were 55.9% for α-PLL and 52.0% for ε-PLL; 40 kDa, 41.2% α-PLL for and 34.7% ε-PLL for; 70 kDa, 28.6% α-PLL for and 29.4% for ε-PLL (see FIG. 6). These values suggest that the porosity of α-PLL and ε-PLL coated microcapsules are similar and thus substrate, nutrients, and waste that are of similar size can traverse each of the AαPA and AεPA membranes.

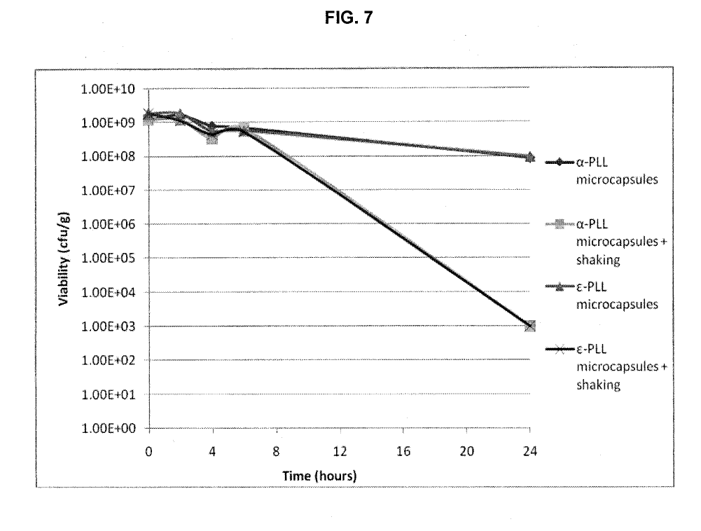

To compare the mechanical stability and physical strength of AαPA versus AεPA microcapsules, a mechanical stability test was designed, based on viability of probiotic cells after 24 h of shaking, using the viability of non-shaken microcapsules as control. The results show no significant difference in the number of viable cells (CFU) remaining in AαPA versus AεPA microcapsules (see FIG. 7). For this reason, the gross mechanical stability of AαPA and AεPA microcapsules appear comparable over a 24-hour period of mechanical disruption.

### Example 2

### Bacteriosatic Effects of Free ε-PLL Versus α-PLL on Probiotic Bacteria and Viability of AαPA Versus AεPA Microencapsulated Probiotic Bacteria

### Bacterial Growth in Presence of α/ε-PLL:

α-poly-L-lysine and ε-poly-L-lysine stock solutions of 0.8% (w/v) were filter sterilized and diluted to the appropriate concentrations (e.g. 0.05% a/ε-PLL (1.25 mL 0.8% (w/v) PLL+18.75 mL MRS). 20 ml MRS was used as control. A 1% (v/v) inoculum (200 μL) of overnight growing culture was added to each 20 ml solution. Incubation was performed at 37° C. and samples were performed at 0, 1, 2, 3, 4, 5, 6, 7, 8, 24 hours. At each time point, 100 μL of sample was removed from the incubating medium for determination of cell viability. Colony forming units (cfu)/ml were measured as indication of cell viability.

### Inhibitory Effect of PLL on Bacterial Growth:

To determine the inhibitory effect of increasing concentrations on bacterial growth of Lactobacillus reuteri NCIMB 701089, 25 mL stock solutions of PLL (1000 μg/mL) were prepared in MRS and filter sterilized with a 0.22 μm syringe filter. In a 96 well plate, serial dilutions from 1000 μg/mL to 62.5 μg/mL were performed in MRS to get a final volume of 150 μL of MRS+PLL in each well. Each concentration was tested in triplicate. An overnight culture was diluted by adding 10 μL in 20 mL of MRS and 15 μL of the diluted cultures were added into each well. Parafilm was used to seal the 96-well plate to prevent any evaporation and the plates were incubated at 37° C. overnight. The plates were read using spectrophotometer at an OD of 620 nm.

Microencapsulated Cell Viability with Secondary Fermentation:

Bacterial cell growth in APA microcapsules was assayed in the presence of α-PLL or ε-PLL to investigate the potential bacteriostatic effect of PLL when electrostatically bound in the membrane of an APA microcapsule. For this, 0.5 g AεPA microcapsules and AαPA microcapsules were weighed out in 50 mL falcon tubes and 10 mL of MRS were added to each falcon tube. Capsules were rinsed at 0, 1, 2, 3, 4, 5, 6, 7, 8, 16, 20, 24 hour time points, with 0.85% (w/v) NaCl diluted in 0.1M sodium citrate (1:20 dilution) and vortexed for release of encased bacteria. Serial dilutions were performed and cell viability was assessed.

### Discussion:

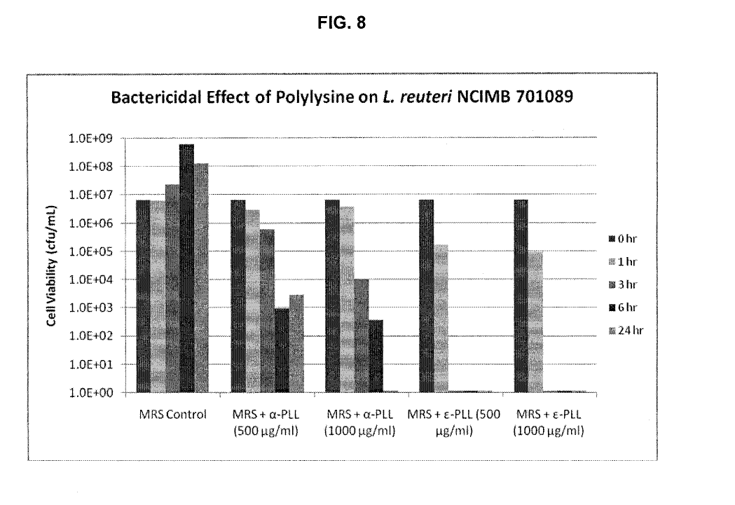

To show the bacteriocidal effect of varying concentrations of α-PLL and ε-PLL, probiotic Lactobacillus reuteri NCIMB 701089 cells were grown in the presence of varying concentrations of α-PLL or ε-PLL (500 μg/ML and 1000 μg/mL) and viability was measure over time (see FIG. 8). The results show that 500 μg/ML and 1000 μg/mL concentrations of both α-PLL and ε-PLL are bactericidal when compared to control, and that the bactericidal effects are greater for ε-PLL than α-PLL against Lactobacillus reuteri NCIMB 701089 in free culture. Accordingly, though ε-PLL has strong anti-microbial properties, it is useful when used as a membrane constituent for the encapsulation of bacteria.

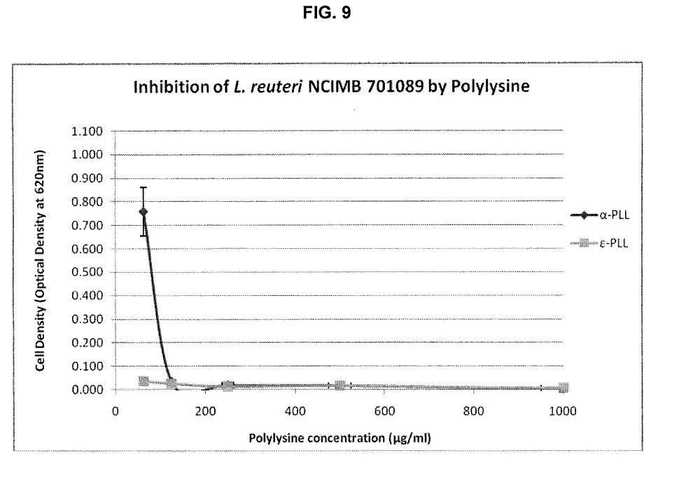

To further show the inhibitory effects of PLL against probiotic Lactobacillus reuteri NCIMB 701089, cells were grown overnight in increasing concentrations of α-PLL or ε-PLL. FIG. 9 shows that both α-PLL and ε-PLL were inhibitory at concentrations above 125 μg/mL, but that only ε-PLL was inhibitory at the lowest concentration tested (62.5 μg/mL). These results confirm that there is an inhibitory effect of ε-PLL on bacterial cell growth at concentrations less than 100 μg/mL and again shows that while ε-PLL has strong anti-microbial properties, it is useful when used as a membrane constituent for the encapsulation of bacteria.

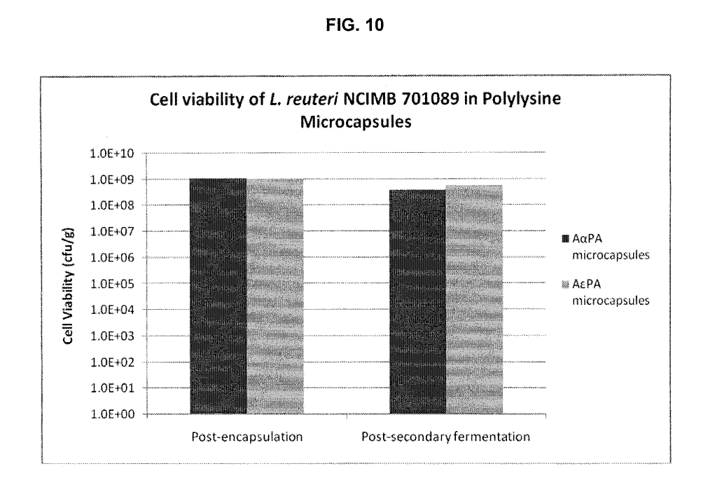

To determine if there was an inhibitory effect of electrostatically bound PLL (bound in the membrane of AαPA or AεPA microcapsules) on probiotic Lactobacillus reuteri NCIMB 701089 cells, bacterial cell viability was measured when grown in MRS media over time (see FIG. 10). The results show that there was no significant inhibitory effect of electrostatically bound α-PLL and ε-PLL and as there was no significant difference in Lactobacillus reuteri NCIBM 701359 cell viability, both immediately after microencapsulation and after 24 hours incubation in MRS media. This result is in stark contrast to the inhibitory effect of ε-PLL at lower concentrations and the inhibitory effect of both α-PLL and ε-PLL at higher concentrations.

### Example 3

### BSH Activity of AαPA Versus AεPA Microencapsulated Lactobacillus Reuteri NCIMB 701089

### Assessment of BSH Activity of Microencapsulated Cells:

To assess the enzymatic activity of α-PLL or ε-PLL microencapsulated bile salt hydrolase (BSH) active probiotic bacteria, BSH activy was determined by HPLC. Microcapsules were washed 3 times with 3 volumes of sterile 0.85% saline in a mesh bottomed beaker to remove all traces of storage media. Once washed, the microcapsules were divided into 2.5 g samples and resuspended in 2 ml MRS. The suspension was added to 18 ml of MRS containing 5 mM taurodeoxycholic acid (TDCA) (Sigma, St Louis) and 5 mM glycodeoxycholic acid (GDCA) (Sigma, St Louis). Samples were taken out at 0.5, 1, 3, and 5 hours after the samples were placed in the 37° C. incubator. The amount of GDCA and TDCA remaining in the samples was analyzed by HPLC. Analyses were performed on a reverse-phase C-18 column (LiChrosorb RP-18 250 nm×4.6 mm, 5 μm) at a flow rate of 1.0 ml/min. The mobile phase was a mixture of methanol and 50 mM sodium acetate buffer (pH4.3 adjusted with o-phosphoric acid) in 70:30 ratio and detection was measured at 210 nm. The BSH activity was evaluated by the amount of deconjugated GDCA and TDCA in samples per hour per gram microcapsules.

### Discussion:

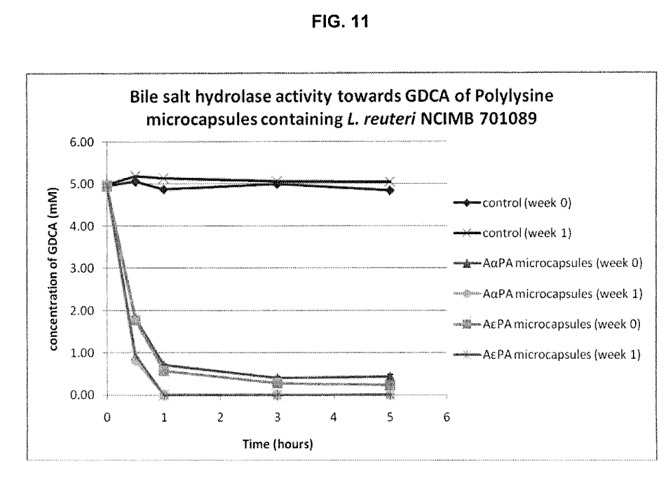

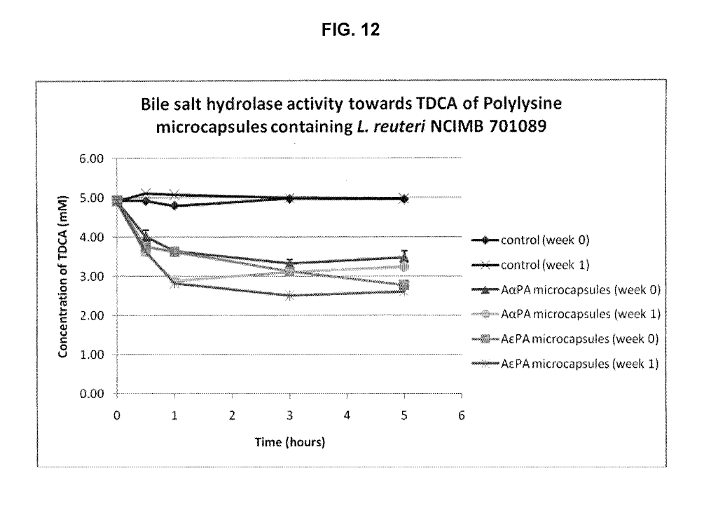

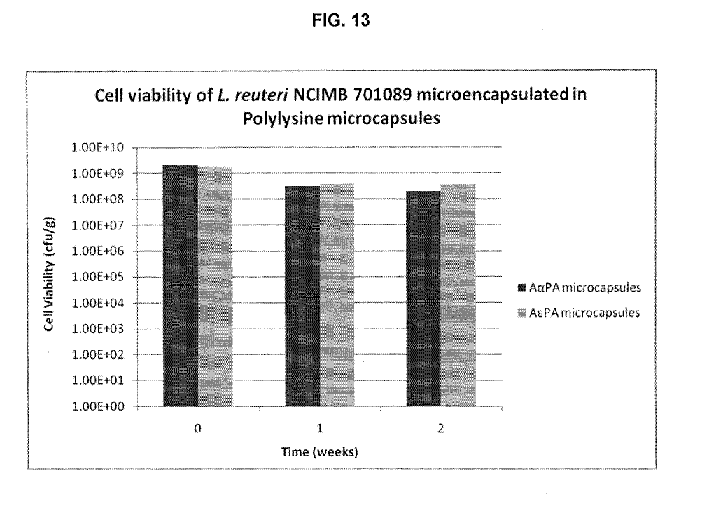

Bile salt hydrolase (BSH) activity of microencapsulated AαPA and AεPA L. reuteri NCIMB 701089 was measured to compare initial enzymatic activity and retention of activity of the microencapsulated probiotic over time. Bile salt hydrolase activity was determined by measuring decreasing concentrations of representative glyco- and tauro-conjugated bile acids (GDCA and TDCA) over time and compared to control. Measurements were taken at 0.5, 1, 3, and 5 h and were analyzed by HPLC (see FIGS. 11 and 12). These results show that there is no significant difference in BSH enzymatic activity between microencapsulated AαPA and AεPA L. reuteri NCIMB 701089 at the time of production and after a week in storage media. Further, the viability of microencapsulated AαPA and AεPA L. reuteri NCIMB 701089 was determined at the time of production and after being stored in 10% MRS solution at 4° C. for a week and there was no significant difference between the two types of microencapsulation (see FIG. 13).

### Example 4

### FAE Activity of AαPA Versus AεPA Microencapsulated Lactobacillus Reuteri NCIMB 5221

### FAE Assay:

The ferulic acid esterase (FAE) activity of Lactobacillus fermentum NCIMB 5221 (Scotland, UK) was carried out by HPLC. Microcapsules were washed 3 times with 3 volumes of sterile 0.85% saline in a mesh bottomed beaker to remove all traces of storage media. Once washed, the microcapsules were divided into 5 g samples and resuspended in 2 ml MRS. A suspension of microcapsules (5 g) were added to 18 ml reaction broth containing 2.2 mM ethyl ferulate in a sterile 50 ml screw-cap Erlenmeyer flask to a final concentration of ethyl ferulate of 2 mM. To prepare the solution of 2 mM ethyl ferulate in MRS (Difco), ethyl ferulate was dissolved in dimethylformamide by 10% w/v and was added to MRS (pH 6.6) drop by drop. The reaction mixture was incubated (37° C.) and samples were taken at 0, 1, 3, and 5 hours. The amount of ferulic acid in samples was analyzed by HPLC. Mobile phase A was 37% methanol, 0.9% acetic acid (v/v) in water. Mobile phase B was 100% methanol. Analyses were performed on a reverse-phase C-18 column (LiChrosorb RP-18 250 nm×4.6 mm, 5 μm) at a flow rate of 1.0 ml/min and detection occurred at 320 nm. The conditions were: Pump A 100% from 0 min to 16 min, linear gradient to 100% pump B from 16 min to 17 min, hold from 17 min to 29 min, linear gradient to 100% pump A from 29 min to 30 min, hold from 30 min to 35 min. The FAE activity was evaluated by the amount of ferulic acid produced per hour per gram microcapsules.

### Discussion:

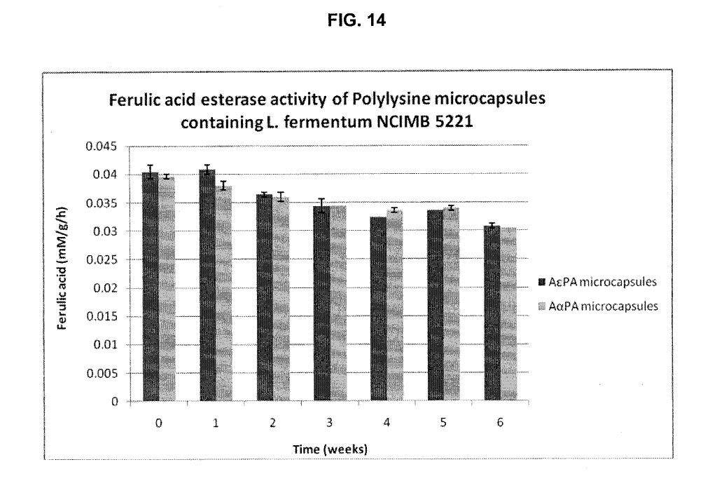

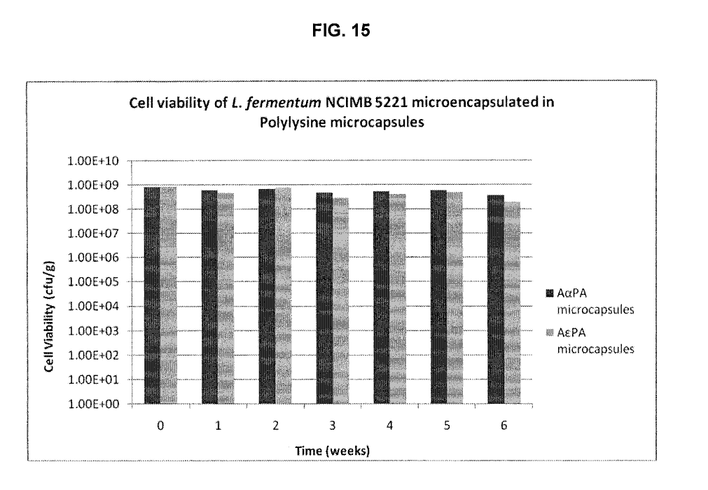

Ferulic acid esterase activity (FAE) activity of microencapsulated AαPA and AεPA L. reuteri NCIMB 5221 was measured to compare initial enzymatic activity and retention of activity of the microencapsulated probiotic over time. FAE activity was determined by measuring the rate of ferulic acid produced per gram microcapsules per hour by AαPA or AεPA microcapsules containing L. fermentum NCIMB 5221. Measurement of FAE activity was determined weekly for a 6 week period and weekly assays measured FAE activity over a 5 hour period. The results show that there was no significant difference in FAE activity between AαPA and AεPA microcapsules containing L. fermentum NCIMB 5221 over the 6 week period (see FIG. 14). Further, viability of microencapsulated AαPA and AεPA microcapsules containing L. fermentum NCIMB 5221 was measured weekly for 6 weeks after storage in 10% MRS solution at 4° C. (see FIG. 15).

### Example 5

### Viability of AαPA Versus AεPA Microencapsulated Mammalian Cells

### THP-1 Cell Encapsulation:

THP-1 cells encapsulated in AεPA microcapsules were examined microscopically and compared with THP-1 cells encapsulated in AαPA microcapsules and THP-1 cells immobilized in alginate beads. THP-1 acute monocytic leukemia cells were obtained from ATCC. The cells were grown to 3-5×10⁵ cells/ml in complete RPMI (Hyclone RPMI+10% Fetal calf serum+0.05 mM 2-mercaptoethanol+penicillin/streptomycine). The cells were pelleted at 800 RPM in 50 ml conical and resuspended at 1.5×10⁶ cells/ml in 1.5% alginate in saline. The cells were immobilized in alginate using an Inotech microencapsulator with a 200 μm nozzle, using 0.1M calcium chloride for gelation. The beads were collected and moved to a 50 ml conical tube in Phosphate buffered saline (PBS, Hyclone) and washed twice by removing the liquid over the settled capsules. The pellet was divided in three equal parts in 15 ml conical tubes and treated with 2 volumes of PBS, 2 volumes of α-poly-L-lysine (0.1% in PBS), or 2 volumes of ε-poly-L-lysine (0.1% in PBS). The capsules were incubated for 20 minutes before washing twice with 2 volumes of PBS. The final coating of the capsules was performed by adding 2 volumes of 0.1% solution of alginate to the α-poly-L-lysine and ε-poly-L-lysine treated capsules. The capsules, and immobilized cells, were washed twice with PBS and 3 times with complete RPMI. The capsules were moved to a 6 well plate in complete RPMI and incubated at 37° C., 5% CO₂ overnight.

### THP-1 Capsules Viability Experiment:

To assess the cell viability of mammalian cells encapsulated in AεPA microcapsules, AαPA microcapsules or alginate beads, the MTT reagent was employed. Capsules containing THP-1 cells were incubated for 4 hours at 37° C., 5% CO₂, in a 24-well plate (0.5 ml capsules/well ×4 replicates) in 1 ml complete RPMI, with 5% MTT reagent solution. After incubation, a solution of 0.1N HCl, 10% SDS was added to each well (1:1) and incubated overnight to allow for the solubilisation of the formazan crystals. Samples of each well (200 μl) were distributed in 4 wells of a 96-well plate and read using a microplate reader. OD570 values are an average of 4 samples, read in triplicate, with standard deviation of the averages.

### Discussion:

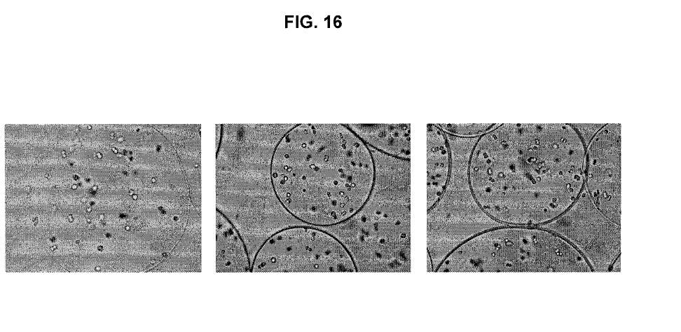

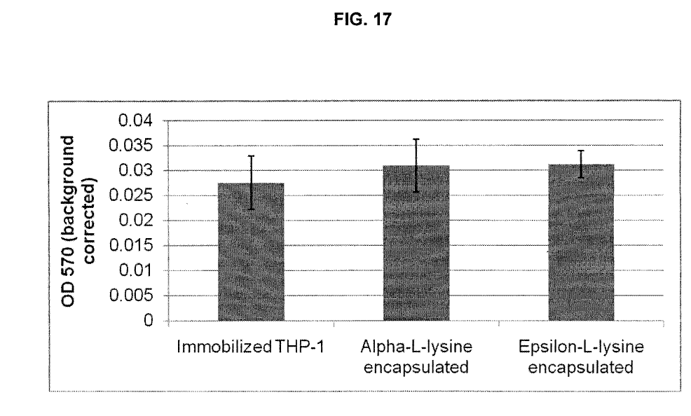

THP-1 monocytic cells were encapsulated in alginate and coated with either α or ε poly-L-lysine to determine the effects of the encapsulation material on the morphology of the capsules and on the viability of the THP-1 cells. FIG. 16 shows the morphological appearance of the immobilized cells, the α-poly-L-lysine encapsulated and ε-poly-L-lysine encapsulated cells. When comparing α and ε capsules, no significant differences were seen between the two membrane types. Viability of the capsules was evaluated using methylthiazol-tetrazolium bromide (MTT). The tetrazolium salt is reduced by metabolically active cells, yielding purple formazan crystals. Using this method, the capsules and immobilized cells were tested after 48 hours of growth in complete RPMI. The reagent was added and the cells incubated for 4 hours. FIG. 17 shows that no significant difference could be seen between the microencapsulated THP-1 while the immobilized cells showed a slight decrease in viability. This may be explained by a more pronounced released of THP-1 cells from the alginate beads in the absence of coating. It thus seems that, overall, both types of coatings will be well suited for use in the encapsulation of mammalian cells.

### Example 6

### AαPA Versus AεPA Microencapsulated Activated Carbon

Activated carbon was encapsulated in AαPA microcapsules and AεPA microcapsules to determine the effects of the encapsulation material on the morphology of the capsules containing activated carbon as a potential haemoperfusion device. Activated carbon (1.0% solution) was first immobilized in alginate using an Inotech microencapsulator with a 400 μm nozzle, using 0.1M calcium chloride for gelation. Alginate microbeads were removed from the CaCl₂ solution and rinsed with 0.85% saline. The beads were divided into two 5 g portions and each portion was suspended in saline for 5 minutes. The washed beads were then coated with 5 mL of 0.1% alpha or 0.1% epsilon PLL solution wherein the capsules were mixed vigorously. The microcapsules were left in the PLL coating solution for 20 minutes with periodic mixing. The PLL solution was removed and the microcapsules were washed for 5 minutes with 0.85% saline. The saline was removed and the microcapsules were re-suspended in a 0.1% alginate coating solution for 20 minutes with mixing. The microcapsules were washed a final time in saline and observed microscopically.

### Discussion:

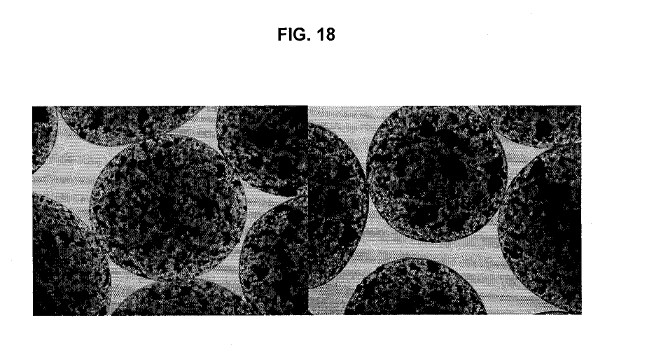

Microencapsulated activated carbon was investigated microscopically to determine whether ε-PLL capsules could be used as a potential haemoperfusion device. FIG. 18 show photomicrographs of activated carbon in AαPA microcapsules (left) or AεPA microcapsules (right). Microscopic evaluation of the microcapsules showed no significant differences indicating that both epsilon and alpha poly-L-lysine can be used as a membrane component for the encapsulation of activated carbon.

### Example 7

### AαPA Versus AεPA Microencapsulated Food Coloring

Elderberry dye as a representative coloring agent was encapsulated in AαPA microcapsules and AεPA microcapsules to determine the effects of the encapsulation material on the morphology of the capsules. A suspension of elderberry coloring agent was made at a concentration of 10% in a 1.75% alginate solution. Microcapsules were made using the procedure described above with a 200 μm nozzle. Alginate microbeads were removed from the CaCl₂ solution and rinsed with 0.85% saline. The beads were divided into two 5 g portions and each portion was suspended in saline for 5 minutes. The saline solution was replaced with 5 mL of 0.1% alpha or epsilon PLL solution and the capsules were mixed vigorously. The microcapsules were left in the PLL coating solution for 20 minutes with periodic mixing. The PLL solution was removed and the microcapsules were washed for 5 minutes with 0.85% saline. The saline was removed and the microcapsules were re-suspended in a 0.1% alginate coating solution for 20 minutes with occasional mixing. The microcapsules were washed a final time in saline and observed microscopically.

### Discussion:

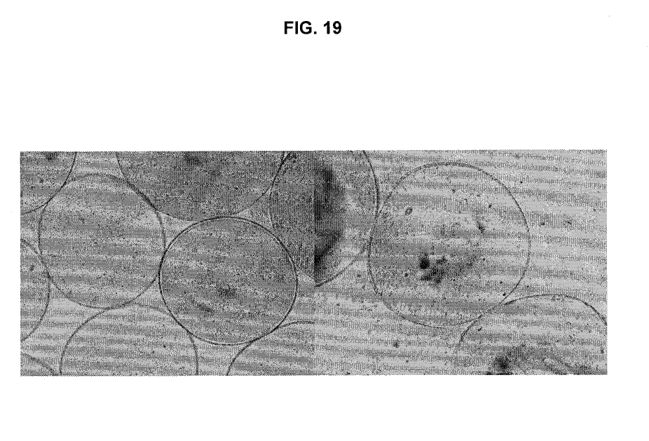

Microencapsulated elderberry dye was investigated microscopically to determine whether ε-PLL capsules could be used for the encapsulation of coloring agents. FIG. 19 show photomicrographs of elderberry coloring agent microencapsulated in AαPA microcapsules (left) or AεPA microcapsules (right).

Microscopic evaluation of the microcapsules showed no significant differences indicating that both epsilon and alpha poly-L-lysine can be used as a membrane component for the encapsulation of elderberry colouring agent.

### Example 8

### AαPA Versus AεPA Microencapsulated Food Matrix (Beet Root)

Beet root as a representative food matrix was encapsulated in AαPA microcapsules and AεPA Microencapsulated to determine the effects of the encapsulation material on the morphology of the capsules. A suspension of lyophilized, milled beet root was made at a concentration of 10 mg/mL in a 1.75% alginate solution. Microcapsules were made using the procedure described above with a 400 μm nozzle. Alginate microbeads were removed from the CaCl₂ solution and rinsed with 0.85% saline. The beads were divided into two 5 g portions and each portion was suspended in saline for 5 minutes. The saline solution was replaced with 5 mL of 0.1% alpha or epsilon PLL solution and the capsules were mixed vigorously. The microcapsules were left in the PLL coating solution for 20 minutes with periodic mixing. The PLL solution was removed and the microcapsules were washed for 5 minutes with 0.85% saline. The saline was removed and the microcapsules were resuspended in a 0.1% alginate coating solution for 20 minutes with occasional mixing. The microcapsules were washed a final time in saline and observed under phase contrast microscopy.

### Discussion:

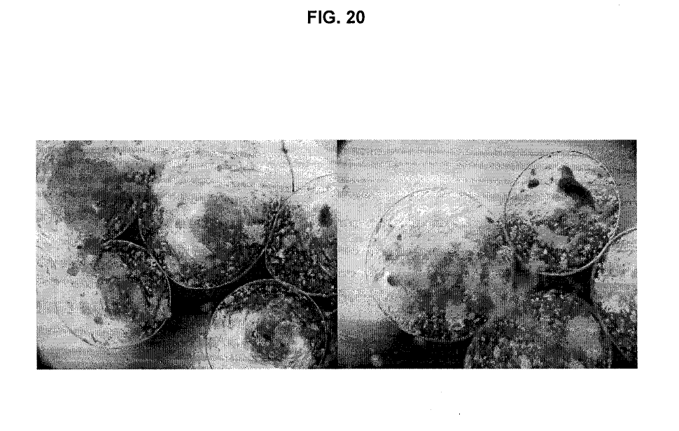

Microencapsulated beet root was investigated microscopically to determine whether ε-PLL capsules could be used for the encapsulation of food matrices. FIG. 20 show photomicrographs of beet root as an example of a food matrix microencapsulated in AαPA microcapsules (left) or AεPA microcapsules (right). Microscopic evaluation of the microcapsules showed no significant differences indicating that both epsilon and alpha poly-L-lysine can be used as a membrane component for the encapsulation of beet root.

### Example 9

### Buoyancy of AαPA Versus AεPA Microcapsules

Alginate microbeads were prepared using an Inotech microencapsulator with a 200 μm nozzle and 0.1M calcium chloride solution for gelation. The alginate microbeads were then removed from the CaCl₂ solution and rinsed with 0.85% saline. The washed microbeads were distributed in 6×10 g aliquots and were re-suspended in 35 ml of 0.85% saline. At t=0, the alginate microbeads (6 aliquots) were completely suspended in solution by shaking and the time to settle prior to PLL coating was observed. The saline was removed and the alginate microbeads were coated with 35 mL of 0.1% alpha (3 aliquots) or 0.1% epsilon (3 aliquots) PLL solution wherein the capsules were mixed vigorously. The microcapsules were left in the PLL coating solution for 15 minutes with periodic mixing. The PLL solution was removed and the microcapsules were washed for 5 minutes with 0.85% saline. The saline was removed and the microcapsules were re-suspended with 35 ml of a 0.1% alginate coating solution for 20 minutes with mixing. The alginate solution was then removed and the AαPA microcapsules (3 aliquots) and AεPA microcapsules (3 aliquots) were re-suspended in 35 ml of 0.85% saline. At t=0, the microcapsules were completely suspended in solution by shaking and the time to settle for each condition was observed. Time to settle prior to and post alpha-PLL coating and epsilon-PLL coating was compared.

To further characterize potential settling differences between AαPA microcapsules and AεPA microcapsules, a glycerol gradient was prepared. Briefly, two sets of serial dilutions of glycerol were prepared in 15 ml conical tubes containing 100%, 66.67%, 44.44%, 29.63% and 19.75% glycerol in 0.85% saline. At t=0, 200 mg of AαPA microcapsules or AεPA microcapsules was added to each glycerol concentration. Time to settle was then noted for each glycerol concentration and compared between AαPA microcapsules and AεPA microcapsules.

### Discussion:

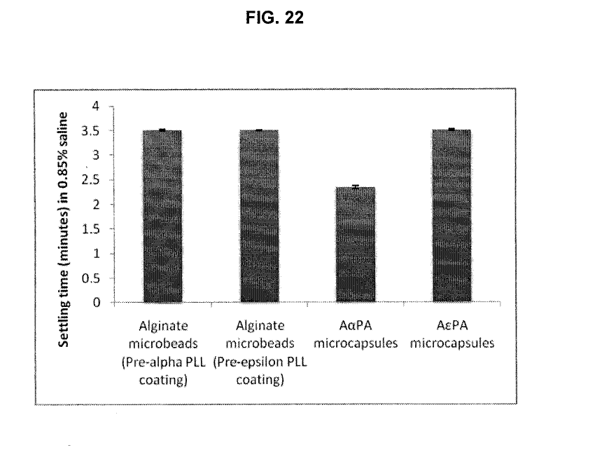

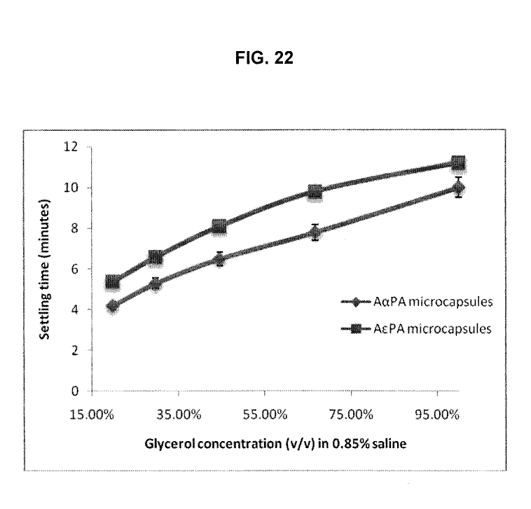

AαPA microcapsules and AεPA microcapsules were investigated for settling time in 0.85% saline solution and a gradient of 100%, 66.67%, 44.44%, 29.63% and 19.75% glycerol in 0.85% saline. FIG. 21 shows the settling time in minutes of alginate microbeads (prior to alpha PLL coating), alginate microbeads (prior to epsilon PLL coating), AαPA microcapsules or AεPA microcapsules in 0.85% saline solution. FIG. 22 shows the settling time in minutes of AαPA microcapsules or AεPA microcapsules in a gradient of 100%, 66.67%, 44.44%, 29.63% and 19.75% glycerol in 0.85% saline. A significantly greater settling time was observed for AεPA microcapsules as compared to AαPA microcapsules indicating that epsilon poly-L-lysine could potentially be used as a membrane component for improved buoyancy, or a decreased tendency to settle, in liquid solutions of varying densities and osmolarities.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

### REFERENCES CITED HEREIN

1. Chang, T. M. & Prakash, S. Artificial cells for bioencapsulation of cells and genetically engineered E. coli. For cell therapy, gene therapy, and removal of urea and ammonia. Methods Mol. Biol. 63, 343-358 (1997).
2. Chang, T. M. S. Semipermeable microcapsules. Science 146, 524-525 (1964).
3. Lim, F. & Sun, A. M. Microencapsulated islets as bioartificial endocrine pancreas. Science 210, 908-910 (1980).
4. Sefton, M. V., May, M. H., Lahooti, S., & Babensee, J. E. Making microencapsulation work: conformal coating, immobilization gels and in vivo performance. J. Control Release 65, 173-186 (2000).
5. Chang, P. L. Encapsulation for somatic gene therapy. Ann. N.Y. Acad. Sci. 875, 146-158 (1999).
6. Prakash, S. & Chang, T. M. In vitro and in vivo uric acid lowering by artificial cells containing microencapsulated genetically engineered E. coli DH5 cells. Int. J. Artif. Organs 23, 429-435 (2000).
7. Prakash, S. & Chang, T. M. Microencapsulated genetically engineered live E. coli DH5 cells administered orally to maintain normal plasma urea level in uremic rats. Nat. Med. 2, 883-887 (1996).
8. Chang T M, & Prakash S. Procedures for microencapsulation of enzymes, cells and genetically engineered microorganisms. Mol. Biotechnol. 17(3): 249-260 (2001).
9. Prakash, S. & Chang, T. M. Growth kinetics of genetically engineered E. coli DH 5 cells in artificial cell APA membrane microcapsules: preliminary report. Artif Cells Blood Substit. Immobil. Biotechnol. 27, 291-301 (1999).
10. Prakash, S. & Chang, T. M. Microencapsulated genetically engineered E. coli DH5 cells for plasma urea and ammonia removal based on: 1. Column bioreactor and 2. Oral administration in uremic rats. Artif. Cells Blood Substit. Immobil. Biotechnol. 24, 201-218 (1996a).
11. Prakash, S. & Chang, T. M. Artificial cell microcapsules containing genetically engineered E. coli DH5 cells for in-vitro lowering of plasma potassium, phosphate, magnesium, sodium, chloride, uric acid, cholesterol, and creatinine: a preliminary report. Artif. Cells Blood Substit. Immobil. Biotechnol. 27, 475-481 (1999a).
12. Lim, F. & Sun, A. M. Microencapsulated islets as bioartificial endocrine pancreas. Science 210, 908-910 (1980).
13. Prakash, S. & Chang, T. M. Microencapsulated genetically engineered E. coli DH5 cells for plasma urea and ammonia removal based on: 1. Column bioreactor and 2. Oral administration in uremic rats. Artif. Cells Blood Substit. Immobil. Biotechnol. 24, 201-218 (1996).
14. Tan, L. H., Chan, L. W., & Heng, P. W. Alginate/starch composites as wall material to achieve microencapsulation with high oil loading. J. Microencapsul. 1-9 (2008).
15. Ouyang, W. et al. Artificial cell microcapsule for oral delivery of live bacterial cells for therapy: design, preparation, and in-vitro characterization. J. Pharm. Pharm. Sci. 7, 315-324 (2004).
16. Tse, M., Uludag, H., Sefton, M. V., & Chang, P. L. Secretion of recombinant proteins from hydroxyethyl methacrylate-methyl methacrylate capsules. Biotechnol. Bioeng. 51, 271-280 (1996).
17. Chang, P. L., Shen, N., & Westcott, A. J. Delivery of recombinant gene products with microencapsulated cells in vivo. Hum. Gene Ther. 4, 433-440 (1993).
18. Koo, J. & Chang, T. M. Secretion of erythropoietin from microencapsulated rat kidney cells: preliminary results. Int. J. Artif. Organs 16, 557-560 (1993).
19. Liu, H. W., Ofosu, F. A., & Chang, P. L. Expression of human factor IX by microencapsulated recombinant fibroblasts. Hum. Gene Ther. 4, 291-301 (1993).
20. Prakash, S. & Chang, T. M. Microencapsulated genetically engineered E. coli DH5 cells for plasma urea and ammonia removal based on: 1. Column bioreactor and 2. Oral administration in uremic rats. Artif. Cells Blood Substit. Immobil. Biotechnol. 24, 201-218 (1996).
21. Chang, T. M. S. Therapeutic applications of polymeric artificial cells. Nature Reviews Drug Discovery 4, 221-235 (2005).
22. Uludag, H., De Vos, P., & Tresco, P. A. Technology of mammalian cell encapsulation. Adv. Drug Deliv. Rev. 42, 29-64 (2000).
23. Chang, T. M. S. Semipermeable microcapsules. Science 146, 524-525 (1964).
24. Chang, T. M. S., Wong, & H. Method for encapsulating biologically active material including cells. U.S. Pat. No. 5,084,350.
25. Zimmerman, U., Cramer, H., Jork, A., & Thurmer, F. Microencapsulation-Based Cell Therapy 548-566 2000).
26. M. Nishikawa and K. Ogawa, “Distribution of microbes producing antimicrobial epsilon-poly-L-lysine polymers in soil microflora determined by a novel method,” 68(7), 3575 (2002).
Ref Type: Journal
27. J. Ouyang, et al., “Production of epsilon-poly-L-lysine by newly isolated Kitasatospora sp. PL6-3,” 1(12), 1459 (2006).
Ref Type: Journal
28. T. Yoshida and T. Nagasawa, “epsilon-Poly-L-lysine: microbial production, biodegradation and application potential,” 62(1), 21 (2003).
Ref Type: Journal

1. A capsule comprising;
i) a core, comprising an active ingredient encapsulated by a first polymer; and
ii) one or more capsular walls surrounding the core, wherein at least one of the capsular walls comprises a second polymer comprising ε-poly-lysine or a derivative thereof.

2. The capsule according to claim 1, wherein the ε-poly-lysine or derivative thereof is produced from the fermentation of yeast or bacteria.

3. The capsule according to claim 2, wherein the ε-poly-lysine produced from the fermentation yeast or bacteria is post-translationally modified.

4. The capsule according to claim 1, wherein the second polymer comprising ε-poly-lysine or a derivative thereof comprises a polymer of the formula (I) 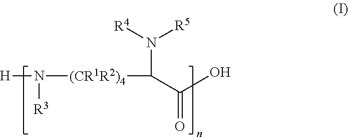
wherein
R¹ and R² are independently or simultaneously selected from H, halo, (C₁-C₁₀)-alkyl, (C₁-C₁₀)-alkoxy, (C₂-C₁₀)-alkenyl, (C₂-C₁₀)-alkynyl, (C₃-C₁₀)-cycloalkyl phenyl, wherein the latter six groups are optionally substituted,
R³ is selected from H, (C₂-C₆)-alkenyl and (C₂-C₆)-alkynyl,
R⁴ and R⁵ are independently selected from H, (C₁-C₁₀)-alkyl, (C₂-C₁₀)-alkenyl, (C₂-C₁₀)-alkynyl, (C₃-C₁₀)-cycloalkyl and phenyl, wherein the latter six groups are optionally substituted,
n is an integer from 1 to 50,
the optional substituents are selected from one to five of halo, C₁₋₆alkyl and fluorosubstituted C₁₋₆alkyl,
and all stereoisomers and enantiomers thereof.

5. (canceled)

6. The capsule according to claim 4, wherein R¹ and R² are independently or simultaneously selected from H and optionally substituted (C₁-C₄)-alkyl.

7. The capsule according to claim 4, wherein R³ is selected from H and (C₁-C₆)-alkyl.

8.-9. (canceled)

10. The capsule according to claim 4, wherein R⁴ and R⁵ are independently selected from H and optionally substituted (C₁-C₄)-alkyl.

11. The capsule according to claim 4, wherein n is an integer from 20 to 40.

12. (canceled)

13. The capsule according to claim 1, wherein the polymer comprising s-poly-lysine or a derivative thereof comprises a polymer of the formula 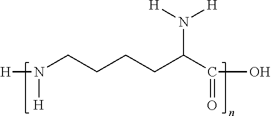
wherein n is an integer from 25 to 30.

14. The capsule according to claim 1, wherein the polymer comprising ε-poly-lysine or derivative thereof comprises a polymer 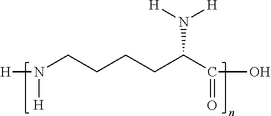
wherein n is an integer from 25 to 30.

15. (canceled)

16. The capsule according to claim 4, wherein the first polymer is alginate and the polymer of the formula (I) is ε-poly-L-lysine.

17. The capsule according to claim 1, wherein the capsule comprises

| | | 1^{st} | 2^{nd} | 3^{rd} | 4^{th} |
|---|---|---|---|---|---|
| | First | capsular | Capsular | Capsular | Capsular |
| Capsule | Polymer | wall | Wall | Wall | Wall |
| 1 | Alginate | ε-poly-l- | Alginate | — | — |
| | | lysine | | | |
| 2 | Alginate | ε-poly-l- | Pectin | ε-poly-l- | Alginate |
| | | lysine | | lysine | |
| 3 | Alginate | Chitosan | Alginate | ε-poly-l- | — |
| | | | | lysine | |
| 4 | Alginate | Chitosan | Alginate | ε-poly-l- | Alginate. |
| | | | | lysine | |

18. The capsule according to claim 1 wherein the capsule has a mechanical stability wherein at least 100 cfu/g (colony forming units/gram) of a probiotic bacteria remain viable after shaking 2.5 g of the capsules containing the bacteria in 10 mL of 10% MRS at 37° C. for 24 hours.

19. The capsule according to claim 1, wherein the active ingredient is a drug, biological material, a fermentation organism, a probiotic organism, inorganic material, a chemical reactant, an adhesive, a food product, a food additive, a colouring agent, an imaging agent or a carbon nanotube.

20. The capsule according to claim 19, wherein the active ingredient is a fermentation organism or probiotic organism, wherein the capsule reduces or prevents phage attack on the organism.

21. The capsule according to claim 1, wherein the capsule is a nanocapsule between 1 nm and less than 1000 nm.

22. The capsule according to claim 1, wherein the capsule is a microcapsule between greater than 1 μm and less than 1000 μm.

23. The capsule according to claim 1, wherein the capsule is a macrocapsule greater than 1000 μm.

24. (canceled)

25. The capsule according to claim 1, wherein the capsule further comprises pharmaceutically acceptable excipients.

26. The capsule according to claim 1, wherein the capsule is produced by extrusion, multi-orifice centrifugation, rotary or spinning atomization, coacervation, interfacial polymerization, pan coating, spray drying, spray chilling, fluid bed coating, solvent evaporation or extraction, sol-gel encapsulation, thermal gelation, stationary nozzle co-extrusion, centrifugal head co-extrusion, submerged nozzle co-extrusion, interfacial polymerization, coacervation or phase separation, matrix polymerization, in-situ polymerization, liposome technology or nanoencapsulation.

27. The capsule according to claim 1, wherein the capsule is suitable for oral administration, topical administration, transplantable, suitable for use in an ex-vivo device or suitable for use in a fermentation process.

28. (canceled)

29. A method for delivering probiotic organisms to the gastrointestinal system of an animal comprising orally delivering a capsule according to claim 1 to the animal, wherein the active ingredient is a probiotic organism.

30. The method according to claim 29, wherein the probiotic organisms are protected from acidic pH, IgA, enzymatic attack, bacteriosins or bacteriophage attack.

31.-33. (canceled)

34. A method for the fermentation of a substrate, comprising contacting the substrate with a capsule of claim 1, wherein the active ingredient comprises microorganisms which ferment the substrate.

35.-41. (canceled)

42. A method for delivering an enzyme or a protein to the gastrointestinal system of an animal comprising orally delivering a capsule according to claim 1 to the animal, wherein the active ingredient comprises an enzyme or a protein.
